SEQUENCE LISTING (5 sequences)

SEQ ID NO: 1 - DNA, 21 bp, Homo sapiens
tatggactccaacctggataa

SEQ ID NO: 2 - DNA, 21 bp, Artificial Sequence
  Description of Artificial Sequence Synthetic primer
tgttccgtttccagcccccaa

SEQ ID NO: 3 - DNA, 21 bp, Artificial Sequence
  Description of Artificial Sequence Synthetic primer
gggcttgtctccgtcgttgat

SEQ ID NO: 4 - DNA, 21 bp, Artificial Sequence
  Description of Artificial Sequence Synthetic primer
cagctcaccatggatgatgat

SEQ ID NO: 5 - DNA, 21 bp, Artificial Sequence
  Description of Artificial Sequence Synthetic primer
ctcggccgtggtggtgaagct

(19) United States
(12) Patent Application Publication
(10) Pub. No.: US 2009/0062222 A1
(43) Pub. Date: Mar. 5, 2009

(54) Methods for Sensitizing Cancer Cells to Inhibitors

(75) Inventors: Michael Sherman, Newton, MA (US); Stuart K Calderwood, Chestnut Hill, MA (US); Nava Zaarur, Brookline, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/088,467

(22) PCT Filed: Sep. 29, 2006
(86) PCT No.: PCT/US2006/038062

§ 371 Date: Sep. 5, 2008

Related U.S. Application Data
(60) Provisional application No. 60/721,800, filed on Sep. 29, 2005.

(51) Int. Cl. A61K 31/704; A61K 31/4375; A61K 31/336; A61K 31/395; A61K 31/69; A61K 31/517; A61K 31/4745; A61P 35/00

(52) U.S. Cl. 514/34; 514/283; 514/450; 514/183; 514/64; 514/267; 514/294

(57) ABSTRACT

The present invention is based on the discovery that inactivation of the heat shock response in cancer cells significantly enhances their sensitivity to proteasome and Hsp90 inhibitors. The inventors have discovered novel compounds which exhibit low toxicity, inhibit the heat shock protein response and sensitize cancer cells to anti-cancer therapies. In general, the heat shock protein inhibitors of the present invention share a common structure, namely a 2H-benzo[a]quinolizine tricyclic ring. Also encompassed are methods for a high throughput screen to identify heat shock inhibitors that sensitize cancer cells to anti-cancer therapies.

### CROSS-REFERENCE TO RELATED APPLICATIONS

This application claims the benefit under 35 U.S.C. §119(e) of U.S. Ser. No. 60/721,800, filed on Sep. 29, 2005, the content of which is herein incorporated by reference in its entirety.

### GOVERNMENT SUPPORT

This invention was made with Government Support under Grant Number CA81244 awarded by the National Institutes of Health. The U.S. Government has certain rights in the invention.

### FIELD OF THE INVENTION

The present invention is directed to a novel set of heat-shock protein inhibitors and to methods of their use in the treatment of cancer. In addition, methods to screen for additional heat-shock protein inhibitors is disclosed.

### BACKGROUND OF THE INVENTION

Cancers are diseases characterized by abnormal, accelerated growth of epithelial cells. This accelerated growth initially causes a tumor to form. Eventually, metastasis to different organ sites can also occur. Although progress has been made in the diagnosis and treatment of various cancers, these diseases still result in significant mortality.

Recently a number of novel anti-cancer therapeutics have been developed that specifically target signaling pathways. Among these newly developed drugs are inhibitors of the proteasome, such as VELCADE® (1), that indirectly activate JNK signaling pathway resulting in apoptosis of cancer cells (2). Proteasome inhibitors were found to be quite potent agents in targeting multiple myeloma, and VELCADE® has already been introduced in clinical practice (3).

Inhibitors of Hsp90, such as geldanomycin, 17-AAG or Radicicol, have also been studied for their anti-cancer activities (4). Since Hsp90 plays a critical role in folding, maturation and stability of several important signaling proteins, including IKK, Act, Raf, and many others, inhibition of Hsp90 by small molecules leads to degradation of these proteins and inactivation of the corresponding signaling pathways (5 6, 7). This, in turn, results in activation of the apoptotic machinery and specific killing of cancer cells. The reason cancer cells are more sensitive to proteasome inhibitors and Hsp90 inhibitors than normal cells is poorly understood. Also, resistant clones emerge occasionally, and the mechanisms of such resistance are unclear. Although both VELCADE® and 17-AAG are well tolerated, the toxicity becomes an issue upon dose escalation. Therefore, there is need for methods that would allow use of lower doses of such compounds in cancer treatment, and thus lower toxicity, while still resulting in effective cancer treatment.

Both proteasome inhibitors and Hsp90 inhibitors are potent inducers of heat shock proteins (Hsps). Since Hsp72, Hsp27 and other Hsps show strong anti-apoptotic potential (8-10, 11), induction of Hsps counterbalances the proapoptotic activities of these drugs, thus leading to enhanced resistance (12, 13). In fact, additional induction of Hsps by pretreatment of cells with mild heat shock led to increased cell resistance to proteasome inhibitors (14). Furthermore, resistance to proteasome inhibitors in a certain lymphoid line was attributed to increased expression of Hsp27 (12). Moreover, inactivation of HSF-1, the main transcription factor that controls induction of Hsps, led to enhanced sensitivity of primary MEF cells to inhibitors of Hsp90 (15).

There have been attempts to target the heat shock response for cancer therapy. In fact, a flavonoid quercetin that inhibits activation of HSF1 was successfully tested as a sensitizer to hyperthermia in animal models (16). However, hyperthermia has a limited potential as a cancer treatment modality, and also quercetin has many side effects in addition to inhibiting Hsp expression. Other attempts to down-regulate the heat shock response during cancer therapy have been unsuccessful.

### SUMMARY OF THE INVENTION

The present invention relates to methods and compositions for sensitizing cancer cells to anti-cancer therapies such that lower dosages of anti-cancer therapies become more effective. For example, the invention provides compositions and methods for the treatment of cancer by administering an effective amount of a heat shock protein inhibitor in combination with an anti-cancer therapy.

We have discovered that the heat shock protein inhibitor agents and compositions of the present invention function to sensitize a cell, that is, make a cancer cell more responsive to an anti-cancer therapy. As used herein a cell that is “more responsive to an anti-cancer therapy” is one where an anti-cancer therapy can be used at a lower dose than the corresponding non-sensitized cell and still result in a similar effect. The cancer cell or cells that are targeted by the methods of the invention can be present in vitro or in vivo. In one embodiment, the cancer cells are present in a mammal, for example a human.

In one embodiment, the heat shock protein inhibitor is administered concurrently with said anti-cancer therapy. Alternatively, the beat shock protein inhibitor is administered prior to said anti-cancer therapy. In an alternative embodiment, the heat shock protein inhibitor is administered after the anti-cancer therapy.

In one embodiment of the present invention the heat shock protein inhibitor is an inhibitor of heat shock protein 72 (Hsp72). In another embodiment, the heat shock protein inhibitor is an inhibitor of heat shock protein 27 (Hsp27).

In general, the heat shock protein inhibitors of the present invention share a common structure, namely a 2H-benzo[a]quinolizine tricyclic ring. Examples of useful heat shock protein inhibitors of the present invention include NZ28 (NCS-134754), emunin (NCS-113238), NZ71, emetine, isocephaeline (NCS-32944), dehydroemetine (NCS-129414), NZ60 (NCS-134757), NZ62 (NCS-134759), NZ61 (NCS-134758), NZ54 (NCS-118072), NZ50 (NCS-10105), tubulosine (NCS-131547), and NZ72 (NCS-131548). Analogs, isomers, metabolites, derivatives, pharmaceutically acceptable salts, pharmaceutical products, hydrates, N-oxides, prodrugs, polymorphs, crystals, or any combination thereof of the above compounds are also encompassed in the present invention.

In one embodiment the heat shock protein inhibitors are selected from terpenoid tetrahydroisoquinoline alkaloids, such as, NZ28 (NCS-134754), NZ71 (emunin; NCS-113238), NZ72 (NCS-131548), dehydroemetine (NCS-129414) and isocephaeline (NCS-32944), or a combination thereof. Analogs, isomers, metabolites, derivatives, pharmaceutically acceptable salts, pharmaceutical products, hydrates, N-oxides, prodrugs, polymorphs, crystals, or any combination thereof are also encompassed.

The novel compositions of the present invention may also be utilized as heat shock protein inhibitors for purposes other than sensitizing cancer cells to anti-cancer therapies. For example, the compositions of the present invention can be used in the prevention and treatment of cancer in general or in the inhibition of viral replication. Such methods are known to those of skill in the art, see, for example, Current Cancer Drug Targets, Volume 3, Number 5, October 2003, pp. 385-390(6) and PCT/US01/27554 (WO-A 2002019965).

In one embodiment of the present invention, the anti-cancer therapy, to be given concurrently or prior to the sensitizing heat shock protein inhibitor(s) of the present invention include heat shock protein 90 (Hsp90) inhibitors or proteasome inhibitors. In one embodiment the HSP90 inhibitor is geldanomycin, 17-AAG or Radicicol. The proteasome inhibitor may be bortezomib (VELCADE®) or MG132 (N-carbobenzoxyl-Leu-Leu-leucinal). Other Hsp90 and proteasome inhibitors are known to those of skill in the art and may be used in the methods of the present invention.

Also encompassed in the present invention is a high throughput screening assay for the discovery and characterization of heat shock protein inhibitors that sensitize cancer cells to anti-cancer agents. This screen is a two step process whereby potential inhibitors are first screened for their ability to inhibit heat shock protein mediated protein refolding. Secondly, compounds that inhibited protein refolding are tested for their ability to inhibit heat shock protein induction by immunoassay, such as immunoblot or activity assay. The second step is essential to ensure that the potential heat shock protein inhibitory compound inhibits heat shock protein induction and not another protein in the protein refolding pathway.

### BRIEF DESCRIPTION OF THE DRAWINGS

FIGS. 1A-1F show the effect of HSF1 depletion on sensitivity of PC-3 cells to heat shock, proteasome and HSP90 inhibitors. FIG. 1A shows depletion of HSF1 by siRNA by immunoblotting. FIG. 1B shows inhibition of Hsp72 induction by a proteasome inhibitor MG132 in cells after depletion of HSF1. FIG. 1C shows that depletion of HSF1 sensitizes cells to apoptosis caused by MG132. FIG. 1D shows quantification of apoptosis measured by PARP cleavage in cells exposed to heat shock, proteasome inhibitor MG132 and Hsp90 inhibitor 17-AAG. This experiment was repeated three times. Quantification of a typical experiment is presented. FIGS. 1E and 1F show the effect of HSF1 depletion on overall clonogenic survival of cells exposed to MG132 (1E) or 17-AAG (1F) for 24 h.

FIGS. 2A-2F show the effect of HSF1 depletion on sensitivity of HCT-116 cells to heat shock, proteasome and HSP90 inhibitors. Infection of HCT-116 cells by retrovirus expressing si-HSF1 was done as described in FIG. 1. FIG. 2A shows the expression of HSF1 in si-HSF1 cells. FIG. 2B shows the expression of Hsp72 in si-HSF1 cells. FIGS. 2C, 2D, and 2E show the effects of HSF1 depletion on sensitivity to apoptosis of cells exposed to heat shock at 45° C. for the indicated time (2C), proteasome inhibitor MG132 (2D), or HSP90 inhibitor Radicicol, (2E) at the indicated concentrations, and PARP cleavage was quantified after overnight incubation by Quantity One software (BIO-RAD) (2F). This experiment was repeated three times. Quantification of a typical experiment is presented.

FIGS. 3A-3E show characterization of Emunin and NZ28. Compounds were added to CHO cells and after 16 hour cells were exposed to heat shock at 45° C. for 10 min. After 6 hours cells were lysed and HSP72 levels were measured by immunoblotting. Control cells (con.) were not exposed to heat shock, and HS con. cells were exposed to heat shock but without compound. As a control for total protein Tubulin antibody was used. FIG. 3A shows the effect of Emunin on induction of Hsp72. FIG. 3B shows a comparison of effects of NZ28 and Quercetin on induction of Hsp72. FIGS. 3C and 3D show that the selected compounds do not affect general protein synthesis. FIG. 3E shows PC-3 cells that were transfected with pGL.hsp70B plasmid, to express luciferase under the regulation of HSP70B gene. Two days after transfection cells were incubated with compounds and exposed to heat shock at 45° C. for 10 min. After overnight incubation luciferase assay was performed. HS control cells were exposed to heat shock without compounds. Control cells weren't expose to HS. FIG. 3F shows PC-cells pre-incubated with Emunin 10 μM or NZ28 2 μM for five hours, and exposed to heat shock at 45° C. for 10 min. One hour after HS cells were lysed, RNA purified, and semi quantitative RT-PCR was performed as described in Materials and Methods. Beta-actin mRNA expression was tested as a control.

FIGS. 4A-4F show Emunin and NZ28 inhibition of HSP72 and HSP27 induction by proteasome and HSP90 inhibitors. MM.1S cells were incubated with proteasome inhibitor VELCADE® or with HSP90 inhibitor Radicicol at the indicated concentrations with or without compounds. 10 μM Emunin or 2 μM NZ28 were added 5 hours before the treatments with the inhibitors. HSP72 and HSP27 levels were measured after overnight incubation. Immunoblotting with anti-tubulin antibody was used as a loading control.

FIGS. 5A-5E show Emunin and NZ28 sensitize MM.1S and PC-3 cells to proteasome and HSP90 inhibitors. In all the cases, compounds were pre-incubated 5 hours before the treatments. Apoptosis was measured by PARP-cleavage. FIG. 5A shows MM.1S cells incubated with 5 nM of proteasome inhibitor VELCADE® with or without 10 μM Emunin. FIG. 5B shows MM.1S cells that were incubated with HSP90 inhibitor Radicicol with or without 10 μM Emunin for 24 hours. FIG. 5C MM.1S cells incubated with 0.1 μM of HSP90 inhibitor Radicicol for 48 h with or without 2 μM NZ28. FIG. 5D shows PC-3 cells that were incubated with 0.13 μM or 0.25 μM of proteasome inhibitor MG132 for 48 h. FIG. 5E shows the effect of Emunin on clonogenic survival of PC-3 cells incubated with 0.5 and 0.25 μM of proteasome inhibitor MG132 for 24 hours or 48 hours, respectively.

### DETAILED DESCRIPTION OF THE INVENTION

We have discovered that inactivation of the heat shock response in cancer cells significantly enhances their sensitivity to anti-cancer agents, such as proteasomne inhibitors and Hsp90 inhibitors.

In one embodiment, the invention provides methods for cancer treatment comprising administering to a subject with cancer cells an effective amount of a heat shock response inactivating agent in combination with an anti-cancer agent. In one embodiment, the addition of heat shock response inactivating agent allows one to reduce the amount of anti-cancer agent compared with a cancer treatment method wherein no heat shock response inactivating agent is used.

We have also discovered novel compounds which exhibit low toxicity, inhibit the heat shock protein response and sensitize cancer cells to anti-cancer therapies. In particular, heat shock protein 72 (Hsp72) and heat shock protein 27 (Hsp27) inhibitors are encompassed. In general, the heat shock protein inhibitors

We have also discovered novel compounds which exhibit low toxicity, inhibit the heat shock protein response and sensitize cancer cells to anti-cancer therapies. In particular, heat shock protein 72 (Hsp72) and beat shock protein 27 (Hsp27) inhibitors are encompassed. In general, the heat shock protein inhibitors of the present invention share a common structure, namely a 2H-benzo[a]quinolizine tricyclic ring.

For example, a compound with the following structure may be used:

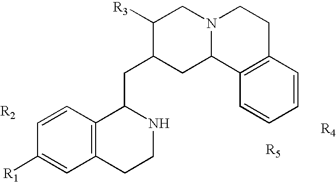

wherein R₁, R₂, R₃, R₄ and R₅ can be selected from —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, R₂, R₃, R₄ and R₅ can be the same or different.

In one embodiment, the structure is:

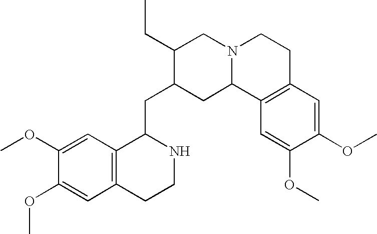

In one embodiment, one uses a compound with the following structure:

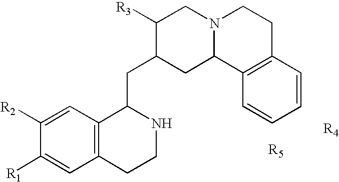

wherein R₁, R₂, R₃, R₄ and R₅ can be selected from —O—(CH₃)ₙ, where n=1-4, —OH, —H, —N—H, S—H, CH₂—CH₃ and C₁-C₄ alkyl, C₁-C₄ alkoxy. C₂-C₄ aryl all of which can be substituted or unsubstituted, substituents include O, N, and S; and R₁, R₂, R₃, R₄ and R₅ can be the same or different.

In one embodiment, the structure is:

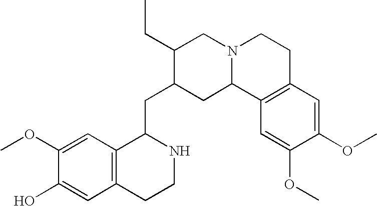

In another embodiment one uses a compound with the following structure:

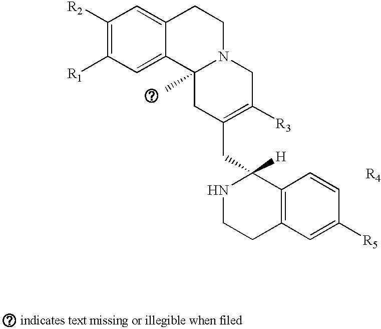

wherein R₁, R₂, R₃, R₄ and R₅ can be selected from —O—(CH₃)ₙ, where n=1-4, —OH, —H, —N—H, S—H, CH₂—CH₃ and C₁-C₄ alkyl, C₁-C₄ alkoxy C₂-C₄ aryl all of which can be substituted or unsubstituted, substituents include O, N, and S; and R₁, R₂, R₃, R₄ and R₅ can be the same or different.

In one embodiment, the structure is:

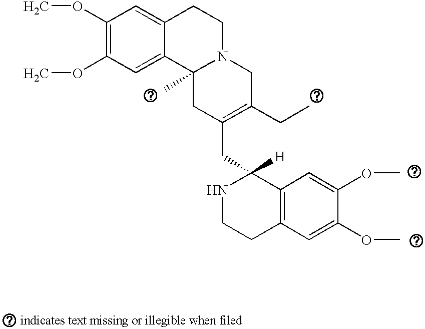

In another embodiment, one uses a compound with the following structure:

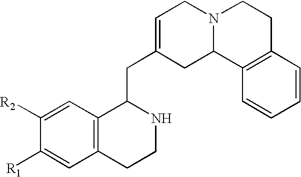

wherein R₁ and R₂, can be selected from —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, and R₂ can be the same or different.

In one embodiment, the structure is:

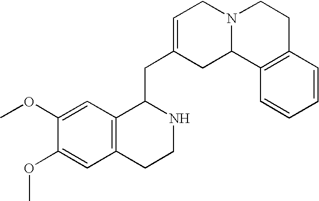

In another embodiment, one uses a compound with the following structure:

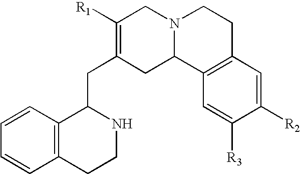

wherein R₁, R₂, and R₃ can be selected from —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, R₂, and R₃ can be the same or different.

In one embodiment, the structure is:

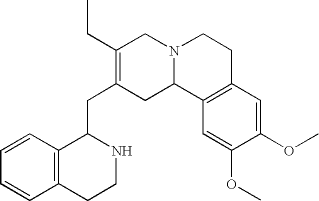

In another embodiment, one uses a compound with the following structure:

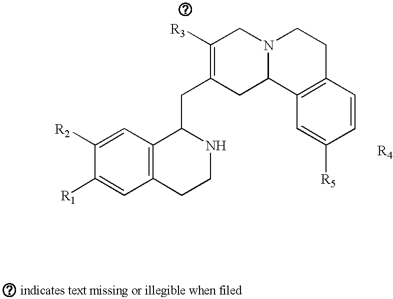

wherein R₁, R₂, R₃′, R₄ and R₅ can be selected from bezyl, —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, R₂, R₃, R₄ and R₅ can be the same or different.

In one embodiment, the structure is:

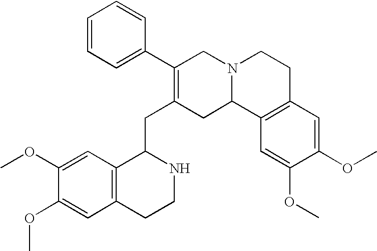

In another embodiment, one uses a compound with the following structure:

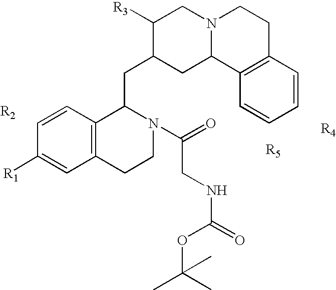

wherein R1, R2, R3, R4, and R5 can be selected from —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, R₂, R₃, R₄ and R₅ can be the same or different.

In one embodiment, the compound is:

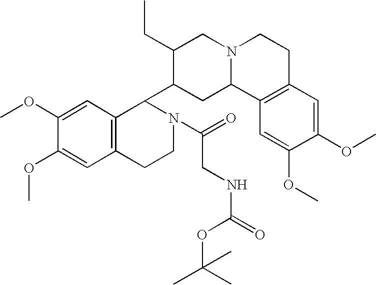

In another embodiment, one uses a compound with the following structure:

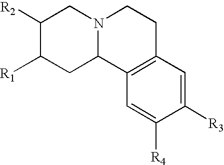

wherein R₁, R₂, R₃ and R₄ can be selected from —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, R₂, R₃, and R₄ can be the sane or different.

In one embodiment, the compound is:

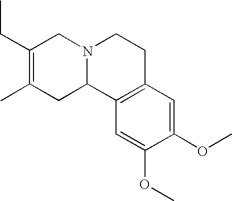

In another embodiment, one uses a compound with the following structure:

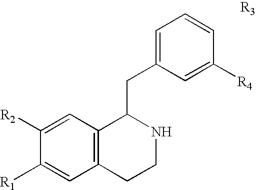

wherein R₁, R₂, R₃, and R₄ can be selected from —O—(CH₃)ₙ, where n=1-4, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S, and R₁, R₂, R₃, and R₄ can be the same or different.

In one embodiment, the structure is:

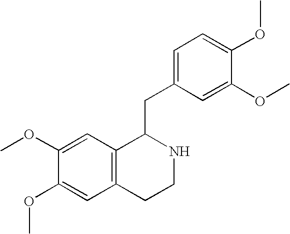

In another embodiment, one uses a compound with the following structure:

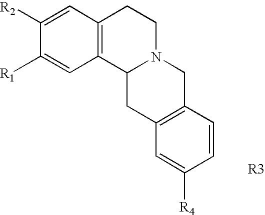

wherein R₁, R₂, R₃, and R₄ can be selected from —O—(CH₃)ₙ, where n=1-4, —OH, —H, —N—H, S—H, CH₂—CH₃ and C₁-C₄ alkyl C₁-C₄ alkoxy, C₂-C₄ aryl all of which can be substituted or unsubstituted, substituents include O, N, and S; and R₁, R₂, R₃, and R₄ can be the same or different.

In one embodiment, the structure is:

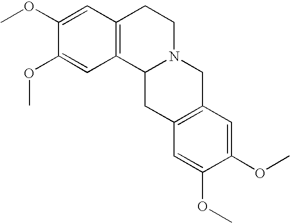

In another embodiment, one uses a compound with the following structure:

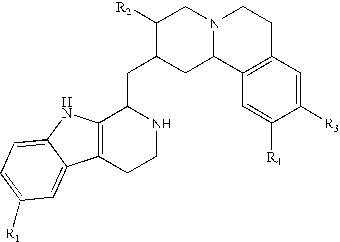

wherein R₁, R₂, R₃, and R₄ can be selected from —O—CH3, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S: and R₁, R₂, R₃, and R₄ can be the same or different.

In one embodiment, the structure is:

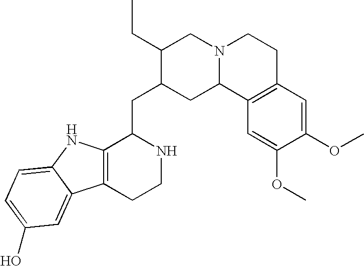

In another embodiment, one uses a compound with the following structure:

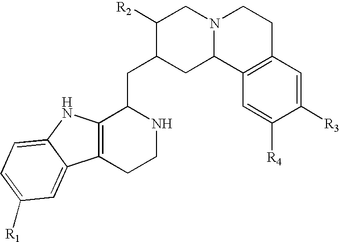

wherein R₁, R₂, R₃, and R₄ can be selected from —O—CH3, —OH, —H, —N—H, S—H, CH₂—CH₃ and C₁-C₄ alkyl, C₁-C₄ alkoxy, C₂-C₄ aryl all of which can be substituted or unsubstituted, substituents include O, N, and S; and R₁, R₂, R₃, and R₄ can be the same or different.

In one embodiment, the structure is:

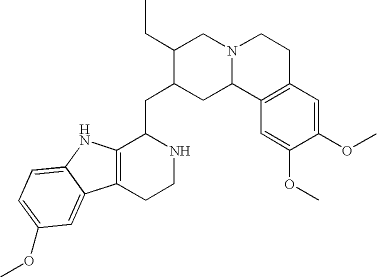

(NCS-134758), NZ54 (NCS-118072), NZ50 (NCS-10105), tubulosine (NCS-131547), and NZ72 (NCS-131548) as shown in Tables 1 and 2 and discussed in the examples. Analogs, isomers, metabolites, derivatives, pharmaceutically acceptable salts, pharmaceutical products, hydrates, N-oxides, prodrugs, polymorphs, crystals, or any combination thereof of the above compounds are also encompassed in the present invention.

In one embodiment, one uses terpenoid tetrahydroisoquinoline alkaloids, such as emetine, klugine, and isocephaeline.

This invention further includes derivatives of the heat shock protein inhibitory compounds. The term “derivatives” includes, but is not limited to, ether derivatives, acid derivatives, amide derivatives, ester derivatives and the like. In addition, this invention further includes hydrates of the heat shock protein inhibitory compounds. The term “hydrate” includes but is not limited to hemihydrate, monohydrate, dihydrate, trihydrate and the like. In addition, metabolites of the heat shock protein inhibitory compounds are encompassed. The term “metabolite” means any substance produced from another substance by metabolism or a metabolic process. Further, pharmaceutical products of the heat shock protein inhibitory compounds are disclosed. The term “pharmaceutical product” means, in one embodiment, a composition suitable for pharmaceutical use (pharmaceutical composition), as described herein. Prodrugs of the heat shock protein inhibitory compounds are disclosed and the term “prodrug” means a substance which can be converted in-vivo into a biologically active agent by such reactions as hydrolysis, esterification, desterification, activation, salt formation and the like. This invention further includes crystals and polymorphs of the heat shock protein inhibitory compounds. The term “crystal” means a substance in a crystalline state. The term “polynorph” refers to a particular crystalline state of a substance, having particular physical properties such as X-ray diffraction, IR spectra, melting point, and the like.

In one embodiment, the compounds used in the methods of the invention do not include geldanamycin (GA)/radicicol (RA)/17-(allylamino)-17-demethoxygeldanamycin (17-AAG, NSC 330507).

In one embodiment, the compounds used in the methods of the invention do not include radicicol (Humicola fuscoatra) an antifungal antibiotic which acts as a Hsp90-specific inhibitor, with chemical formula C₁₈H₁₇ClOC₆ and CAS No. [12772-57-5].

In one embodiment, the compounds used in the methods of the invention do not include a flavonoid quercetin.

Methods for Treating a Patient

In one embodiment, the invention provides a method for treatment for a patient affected by or at risk for developing cancer by administering to the patient a combination treatment comprising a heat shock protein inhibitor and an anti-cancer therapy.

In one embodiment, the heat shock protein inhibitor exhibits low toxicity, inhibits the heat shock protein response and sensitizes cancer cells to anti-cancer therapies.

In one embodiment, the heat shock proteins inhibited are heat shock protein 72 (Hsp72) and heat shock protein 27 (Hsp27).

In general, the heat shock protein inhibitors of the present invention share a common structure, namely a 2H-benzo[a]quinolizine tricyclic ring.

The treatment comprised the administration of a heat shock protein inhibitor. The treatment may involve a combination of treatments, including, but not limited to a heat shock protein inhibitors in combination with other heat shock protein inhibitor, chemotherapy, radiation, etc.

Thus, in connection with the administration of a beat shock protein inhibitor, an inhibitor which sensitizes a cancer cell to an anti-cancer therapy indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition. The beneficial effect is greater than would be expected from administering an anti-cancer therapy alone.

In one embodiment of the present invention, the anti-cancer therapy, to be given concurrently or prior to the sensitizing heat shock protein inhibitor(s) of the present invention comprises heat shock protein 90 (Hsp90) inhibitors or proteasome inhibitors. In one embodiment, the HSP90 inhibitor is geldanomycin, 17-AAG or Radicicol. The proteasome inhibitor may be bortezomib (VELCADE®) or MG132 (N-carbobenzoxyl-Leu-Leu-leucinal). Other Hsp90 and proteasome inhibitors are known to those of skill in the art and may be used in the methods of the present invention.

In another embodiment, the anti-cancer agent is a chemotherapeutic agent. In another embodiment, the anti-cancer agent is a radiotherapy. In yet another embodiment, the anti-cancer therapy is antiangiogenic therapy (e.g., endostatin, angiostatin, TNP-470, Caplostatin (See, for example, Stachi-Fainaro et al., Cancer Cell 7(3), 251 (2005)). Combinations, such as radiotherapy and chemotherapeutic agent or chemotherapy and antiangiogenic therapy, or radiation therapy and antiangiogenic therapy may also be used as well as combinations of the agents such as chemotherapeutic agents in combination with the heat shock inhibiting agents of the present invention.

The anti-cancer agents of the present invention may be, for example, therapeutic radionuclides, drugs, hormones, hormone antagonists, receptor antagonists, enzymes or proenzymes activated by another agent, autocrines, cytokines or any suitable anti-cancer agent known to those skilled in the art. In one embodiment, the anti-cancer agent is AVASTIN®, an anti-VEGF antibody proven successful in anti angiogenic therapy of cancer against both solid cancers and hematological malignancies. See, e.g., Ribatti et al. 2003 J Hematother Stem Cell Res. 12(1), 11-22. Toxins also can be used in the methods of the present invention. Other therapeutic agents useful in the present invention include anti-DNA, anti-RNA, radiolabeled oligonucleotides, such as antisense oligonucleotides, anti-protein and anti-chromatin cytotoxic or antimicrobial agents. Other therapeutic agents are known to those skilled in the art, and the use of such other therapeutic agents in accordance with the present invention is specifically contemplated.

The anti-cancer agent may be one of numerous chemotherapy agents such as an alkylating agent, an antimetabolite, a hormonal agent, an antibiotic, an antibody, an anti-cancer biological, gleevec, colchicine, a vinca alkaloid, L asparaginase, procarbazine, hydroxyurea, mitotane, nitrosoureas or an imidazole carboxamide. Suitable agents are those agents that promote depolarization of tubulin or prohibit tumor cell proliferation. Chemotherapeutic agents contemplated as within the scope of the invention include, but are not limited to, anti-cancer agents listed in the Orange Book of Approved Drug Products With Therapeutic Equivalence Evaluations, as compiled by the Food and Drug Administration and the U.S. Department of Health and Human Services. Non-limiting examples of chemotherapeutic agents include, e.g., carboplatin and paclitaxel.

The anti-cancer agent to be combined with the heat shock protein inhibitor of the present invention may be a chemotherapeutic agent. Chemotherapeutic agents are known in the art and include at least the taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be one or more agents chosen from the non-limiting group of steroids, progestins, estrogens, antiestrogens, or androgens. Even more specifically, the chemotherapy agents may be azaribine, bleomycin, bryostatin-1, busulfan, carmustine, chlorambucil, carboplatin, cisplatin, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, paclitaxel, phenyl butyrate, prednisone, procarbazine, semustine streptozocin, tamoxifen, taxanes, taxol, testosterone propionate, thalidomide, thioguanine, thiotepa, uracil mustard, vinblastine, or vincristine. The use of any combination of chemotherapy agents is also contemplated. The administration of the chemotherapeutic agent may be before, during or after the administration of a sensitizing heat shock protein inhibitor.

Other suitable anti-cancer agents are selected from the group consisting of radioisotope, boron addend, immunomodulator, toxin, photoactive agent or dye, cancer chemotherapeutic drug, antiviral drug, antifungal drug, antibacterial drug, antiprotozoal drug and chemosensitizing agent (See, U.S. Pat. Nos. 4,925,648 and 4,932,412). Suitable chemotherapeutic agents are described in REMINGTON'S PHARMACEUTICAL SCIENCES, 19th Ed. (Mack Publishing Co. 1995), and in Goodman and Gilman's The Pharmacological Basis of Therapeutics (Goodman et al., Eds. Macmillan Publishing Co., New York, 1980 and 2001 editions). Moreover, a suitable therapeutic radioisotope can be selected from the group consisting of α-emitters, β-emitters, γ-emitters, Auger electron emitters, neutron capturing agents that emit α-particles and radioisotopes that decay by electron capture. Preferably, the radioisotope is selected from the group consisting of ²²⁵Ac, ¹⁹⁸Au, ³²P, ¹²⁵I, ¹³¹I, ⁹⁰Y, ¹⁸⁶Re, ¹⁸⁸Re, ⁶⁷Cu, ¹⁷⁷Lu, ²¹³Bi, ¹⁰Bi, and 211At.

Where more than one therapeutic agent is used, they may be the same or different. For example, the therapeutic agents may comprise different radionuclides, or a drug and a radionuclide.

The compounds of the invention are preferably used in combination with the anti-cancer treatment. However, in one embodiment, one uses the compounds of the invention by itself.

The compounds of this invention can be administered by oral, parenteral (intramuscular (i.m.), intraperitoneal (i.p.), intravenous (i.v.) or subcutaneous (s.c.) injection), nasal, vaginal, rectal or sublingual routes of administration as well as intrapulmonary inhalation can be formulated in dose forms appropriate for each route of administration. The compounds of the invention may also be administered using a catheter or injection directly to the organ or tissue needing anti-cancer treatment or to the tumor mass or cells

Solid dose forms for oral administration include capsules, tablets, pills, powders and granules. In such solid dose forms, the active compound is mixed with at least one inert carrier such as sucrose, lactose, or starch. Such dose forms can also comprise, as is normal practice, additional substances other than inert diluents, e.g., lubricating agents such as magnesium stearate. In the case of capsules, tablets and pills, the dose forms may also comprise buffering agents. Tablets and pills can additionally be prepared with enteric coatings.

Liquid dose forms for oral administration include emulsions, solutions, suspensions, syrups, the elixirs containing inert diluents commonly used in the art, such as water. Besides, such inert diluents, compositions can also include adjuvants, such as wetting agents, emulsifying and suspending agents, and sweetening, flavoring, and perfuming agents.

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dose forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in a medicum of sterile water, or some other sterile injectable medium immediately before use.

The amount of the Hsp inhibiting agents or combination of compounds of the present invention administered will vary depending on numerous factors, e.g., the particular animal treated, its age and sex, the desired therapeutic affect, the route of administration and which polypeptide or combination of polypeptides are employed. In all instances, however, a dose effective (therapeutically effective amount) to promote release and elevation of growth hormone level in the blood of the recipient animal is used. Ordinarily, this dose level falls in the range of between about 0.1 μg to 10 μg of total compound per kg of body weight. The preferred amount can readily be determined empirically by the skilled artisan based upon the present disclosure.

When the mode of administration is oral, greater amounts are typically needed. The exact level can readily be determined empirically based upon the skill in the art of cancer treatments.

In general, as aforesaid, the administration of combinations of the Hsp27 or Hsp72 heat shock protein inhibiting compounds will allow for lower doses of the anti-cancer treatments or compounds to be employed relative to the dose levels required for individual anti-cancer treatments or compounds in order to obtain a similar response, due to the sensitizing effect of the Hsp inhibition to the cancer cell response to other anti-cancer treatments.

Also included within the scope of the present invention are compositions that comprise, as an active ingredient, the organic and inorganic addition salts of the above-described polypeptides and combinations thereof; optionally, in association with a carrier, diluent, slow release matrix, or coating.

The organic or inorganic addition salts of the compounds and combinations thereof contemplated to be within the scope of the present invention include salts of such organic moieties as acetate, trifluoroacetate, oxalate, valerate, oleate, laurate, benzoate, lactate, tosylate, citrate, maleate, fumarate, succinate, tartrate, naphthalate, and the like; and such inorganic moieties as Group I (i.e., alkali metal salts), Group II (i.e. alkaline earth metal salts) ammonium and protamine salts, zinc, iron, and the like with counterions such as chloride, bromide, sulfate, phosphate and the like, as well as the organic moieties referred to above.

Pharmaceutically acceptable salts are preferred when administration to human subjects is contemplated. Such salts include the non-toxic alkali metal, alkaline earth metal and ammonium salts commonly used in the pharmaceutical industry including sodium, potassium, lithium, calcium, magnesium, barium, ammonium and protamine salts which are prepared by methods well known in the art. The term also includes non-toxic acid addition salts which are generally prepared by reacting the compounds of this invention with a suitable organic or inorganic acid. Representative salts include hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, napthylate and the like.

High Throughput Screen

Also encompassed in the present invention is a high throughput screen to identify compounds that inhibit the heat shock response. As discussed herein and in the examples, the screen is carried out in two phases. The first phase identifies compounds that inhibit heat shock protein mediated protein refolding. The second phase further screens the compounds identified in the first phase for their ability to specifically inhibit heat shock protein induction. The second step is essential to ensure that the compounds specifically target heat shock protein induction.

In particular, a two-stage high throughput screen for inhibitors of Hsp expression encompasses a first stage and a second stage. The first stage involves a cell-based screen for Hsp-mediated refolding of heat-denatured proteins. In one embodiment, Hsp-mediated refolding of heat-denatured luciferase is disclosed. Firefly luciferase expressed in mammalian cells is very sensitive to denaturation upon exposure of cells to severe but non-lethal heat insults. On the other hand, pretreatment of cells with milder heat shock leads to induction of Hsps which protect luciferase from further exposure to denaturing heat insults, and facilitates luciferase refolding. Therefore, exposure of cells to a potential inhibitor of Hsp induction will suppress the protective effects of mild heat shock, and result in reduced luciferase activity after the second denaturing insult. While luciferase is one example, any protein known to be sensitive to heat denaturation and renaturation may be used in the present method. Also included are derivatives of luciferase, including protein fragments, isomers, and mutated derivatives.

In one embodiment of the high throughput assay, cells, such as CHO cells, expressing a reporter gene, such as luciferase or a fluorescent green protein or the like, under the control of an inducible or repressible promoter, such as tetracycline or ecdosyne inducible or repressible promoter are utilized.

For example in a system wherein a tetracycline-off-type promoter is used, the cells seeded in cell growth containers, such as cell growth vials, chambers, plates, multi-well plates, three-dimensional cell growth matrixes, and such, with medium that lacks tetracycline are allowed to express a reporter gene, such as luciferase, GFP or a like reporter gene. The test compounds are added after at least about 1, 2, 3, 4, 5, 6, 7, 8, 9, 10, or up to at least 24 hours or even longer. In one embodiment, one adds the compounds after about 4-9 hours. Following incubation at the normal growth temperature of the cells for at least about 1, 2, 3, 4, 5, 6, 7, 8, 9, 10, or up to 24 hours or longer, the cells are exposed to a heat shock. The heat shock can be induced using any temperature above the normal growth temperature of the cells. For example, if the normal growth temperature of the cells is 37° C., one can use temperatures such as about 38° C., 39° C., 40° C., 41° C., 42° C., 43° C., 44° C., 45° C., 46° C., 47° C., 48° C., 49° C., 50° C. or even at higher temperatures to induce a heat shock. Typically, one uses the higher temperature for several minutes to maximum of several hours. For example, time that the cells are exposed to the elevated temperature may be about 2, 3, 4, 5, 6, 7, 8, 9, 10, 11, 12, 13, 14, 15, 16, 17, 18, 19, 20, 20-30, 30-60 minutes or even longer. Preferably short tines, such as 2-15 minutes are used. The temperature and timing can be varied according to protocols and standard practices known to those of skill in the art.

In one embodiment, cells are grown at about 37° C. for several hours, and exposed to heat shock at about 45° C. for about 10 min to induce production of heat shock proteins, Hsps.

After an additional incubation at the normal growth temperature of the cells, such as about 37° C., the cells are exposed to a severe denaturing heat shock. A severe denaturing heat shock is typically performed at the same temperature as the heat shock but for a longer period of time. Typically one uses temperatures, that does not permanently lead to the reporter protein denaturation. For example, temperatures at about 40, 41, 42, 43, 44, 45, 46, 47, 48, 49, or 50° C. can be used for at least 20-30, 40, 50, 60, minutes or up to about 2-4 hours.

In one embodiment, the cells are exposed to a severe heat shock at about 45° C. for about 50 min, followed by recovery for about 70 min at about 37° C. to allow reporter protein, such as luciferase, GFP or a like, refolding. As will be expected, the exact temperatures and times may be altered as it is known to those of skill in the art. The cells are then assayed for the reporter gene activity. Exposure of cell to a “severe heat shock” without pretreatment with “mild heat shock” typically leads to unrepairable damage of the reporter protein. On the other hand, induction of Hsps after mild heat shock allows rapid refolding of the reporter proteins. For example, severe heat shock after a mild heat shock, results in about 50% of properly folded luciferase protein after 70 min of recovery. Compounds that inhibit induction of Hsps must prevent reporter protein refolding, and to select the inhibitors a cut-off line of inhibition of the reporter protein activity is established. This cut-off may be determined by the skilled practitioner to allow for an increase or decrease in the sensitivity of the assay as is known to those of skill in the art.

In addition, a counter-screen against toxic chemicals may be employed. In this embodiment compounds are added to cells in cell growth vessels such as plates, multi-well plates, vials, chambers, and the like, and kept for the duration of the entire experiment at the normal cell growth temperature, for example, at about 37° C. without exposure to a mild or a severe heat shock. The cut-off line for toxicity may be about 20%, 21%, 22%, 23%, 24%, 25%, 26%, 27%, 28%, 29%, 30%, 31%, 32%, 33%, 34%, 35%, or up to 35-40% inhibition of the reporter protein, such as luciferase, activity in cells kept at the normal cell growth temperature, such as 37° C. However, the cut-off line for toxicity may be altered as determined by the skilled practitioner.

In the second phase of the screen, compounds selected in the first step are directly tested for inhibition of Hsp induction. In one embodiment this screen is an immunoassay. In particular an immunoblot (e.g. Western Blot) may be utilized. For example, the compounds identified in the first phase of the screen may be contacted with the cells and the cells may be screened with an anti-Hsp72 antibody to determine if the heat shock response has been inhibited. It should be recognized that other immunoassays, known to those of skill in the art, may be utilized. In particular, antibody techniques such as immunohistochemistry, immunocytochemistry, FACS scanning, immunoblotting, radioimmunoassays, western blotting, immunoprecipitation, enzyme-linked immunosorbant assays (ELISA), and derivative techniques that make use of antibodies directed against activated heat shock proteins may be utilized.

Immunohistochemistry (“IHC”) and immunocytochemistry (“ICC”) techniques, for example, may be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody. Immunoshistochemical assays are known to those of skill in the art (e.g., see Jalkanen, et al., J. Cell. Biol. 101:976-985 (1985); Jalkanen, et al., J. Cell. Biol. 105:3087-3096 (1987).

Antibodies, polyclonal or monoclonal, can be purchased from a variety of commercial suppliers, or may be manufactured using well-known methods, e.g., as described in Harlow et al., Antibodies: A Laboratory Manual, 2nd Ed; Cold Spring Harbor Laboratory Press, Cold Spring Harbor, N.Y. (1988). In general, examples of antibodies useful in the present invention include anti-heat shock protein antibodies, such as, hsp27 and hsp72. Such antibodies can be purchased, for example, from Upstate Biotechnology (Lake Placid, N.Y.), New England Biolabs (Beverly, Mass.), NeoMarkers (Fremont, Calif.)

Immunological methods of the present invention are advantageous because they require only small quantities of biological material. Such methods may be done at the cellular level and thereby necessitate a minimum of one cell. Preferably, several cells are obtained and assayed according to the methods of the present invention.

Accordingly, in one embodiment the invention provides a method for sensitizing a cancer cell to an anti-cancer therapy comprising: administering to said cancer cells an effective amount of a heat shock protein inhibitor and an anti-cancer therapy.

In one embodiment, the heat shock protein inhibitor is an inhibitor of heat shock protein 72 (Hsp72) or heat shock protein 27 (Hsp27).

In one embodiment, the heat shock protein inhibitor contains a 2H-benzo[a]quinolizine tricyclic ring.

In one embodiment, the heat shock protein inhibitor is selected from the group consisting of NZ28 (NCS-134754), emunin (NCS-113238), NZ71, emetine, isocephaeline (NCS-32944), dehydroemetine (NCS-129414), NZ60 (NCS-134757), NZ62 (NCS-134759), NZ61 (NCS-134758), NZ54 (NCS-118072), NZ50 (NCS-10105), tubulosine (NCS-131547), and NZ72 (NCS-131548).

In one embodiment, the anti-cancer therapy is selected from the group consisting of inhibitors of heat shock protein 90 (HSP90) or proteasomne inhibitors.

In one embodiment, the inhibitor of heat shock protein 90 (HSP90) is selected from the group consisting of geldanomycin, 17-AAG and radicicol.

In one embodiment, the proteasome inhibitor is bortezomib (VELCADE®) or MG132 (N-carbobenzoxyl-Leu-Leu-leucinal).

In one embodiment, the targeted cancer cells are in vivo.

In one embodiment, the heat shock protein inhibitor is administered concurrently with said anti-cancer therapy.

In one embodiment, the heat shock protein inhibitor is administered prior to said anti-cancer therapy

In one embodiment, the invention provides a use of a composition for the inhibition of heat shock protein comprising a compound containing a 2H-benzo[a]quinolizine tricyclic ring to sensitize a malignant cell to administration of an anticancer agent. In one embodiment, the composition comprises NZ28 (NCS-134754). In one embodiment, the composition comprises emunin (NCS-113238).

In another embodiment, the invention provides a use of the composition comprising 2H-benzo[a]quinolizine tricyclic ring, wherein one can administer at least about 5-10%, 10-20%, 20%, or about 25-50% less of an anti-cancer agent to obtain a similar result as an isogenic malignant cell without administration of the compound.

In one embodiment, the compositions comprising 2H-benzo[a]quinolizine tricyclic ring are selected from the group consisting of NZ28 (NCS-134754), emunin (NCS-113238), NZ71, emetine, isocephaeline (NCS-32944), dehydroemetine (NCS-129414), NZ60 (NCS-134757), NZ62 (NCS-134759), NZ61 (NCS-134758), NZ54 (NCS-118072), NZ50 (NCS-10105), tubulosine (NCS-131547), and NZ72 (NCS-131548).

In another embodiment, the invention provides a method for detecting and assaying compounds with heat shock protein inhibitory activity, comprising: a) contacting cells expressing a reporter gene with a compound; b) exposing said cells to mild heat shock to induce heat shock protein expression; c) allowing said cells to incubate for at least two hours; d) exposing said cells to denaturing heat shock; e) allowing said cells to incubate at a normal growth temperature of the cells; f) assaying said cells for the reporter protein activity; g) selecting a compound wherein said reporter protein activity is inhibited compared to a control sample that did not receive said compound; and h) further screening said selected compound for its ability to inhibit heat shock protein induction, wherein said further screen comprises an immunoassay for one or a plurality of heat shock proteins.

In one embodiment, the invention provides a high throughput screen for detecting and assaying compounds with heat shock protein inhibitory activity, comprising: a) contacting cells expressing luciferase with a compound; b) exposing said cells to mild heat shock to induce heat shock protein expression, wherein said mild heat shock is about 45 degrees Celsius for about 10 minutes; c) allowing said cells to incubate for at least two hours; d) exposing said cells to denaturing heat shock, wherein said denaturing heat shock is about 45 degrees Celsius for about one hour; e) allowing said cells to incubate for about one hour at 37 degrees Celsius; f) assaying said cells for luciferase activity; g) selecting compounds wherein said luciferase activity is inhibited compared to a control sample that did not receive said compound; and h) further screening said selected compounds for their ability to inhibit heat shock protein induction, wherein said further screen comprises an immunoassay for one or a plurality of heat shock proteins.

In one embodiment, the mild heat shock is about 45 degrees Celsius for about 10 minutes.

In one embodiment, the denaturing heat shock is about 45 degrees Celsius for about one hour.

In one embodiment, the normal growth temperature of the cells in step e) is about 37 degrees Celsius and the incubation time is about one hour.

In one embodiment, the immunoassay is an immunoblot.

In one embodiment, the immunoassay is an ELISA.

In one embodiment, the immunoassay is performed to detect a heat shock protein selected from the group consisting of heat shock protein 72 and heat shock protein 27.

The invention will be further illustrated by the following non-limiting examples.

### EXAMPLES

Inhibition of the heat shock response sensitizes cancer cells to proteasome and Hsp90 inhibitors.

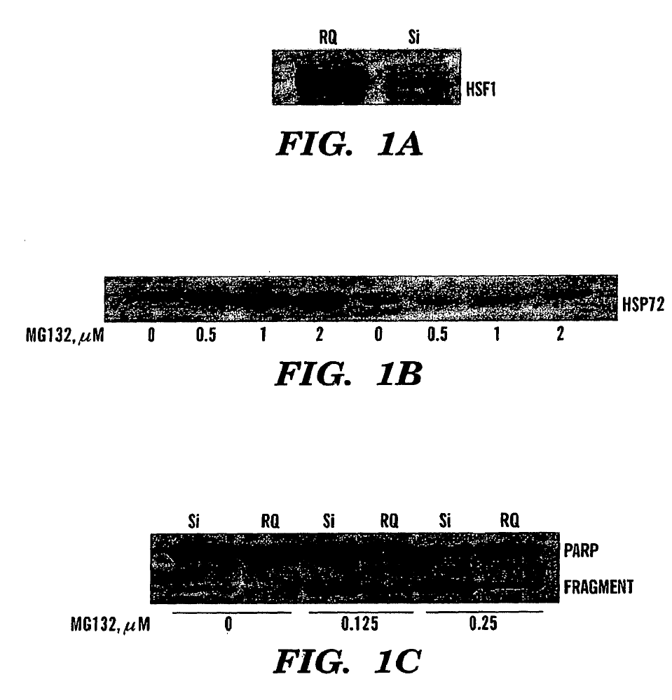

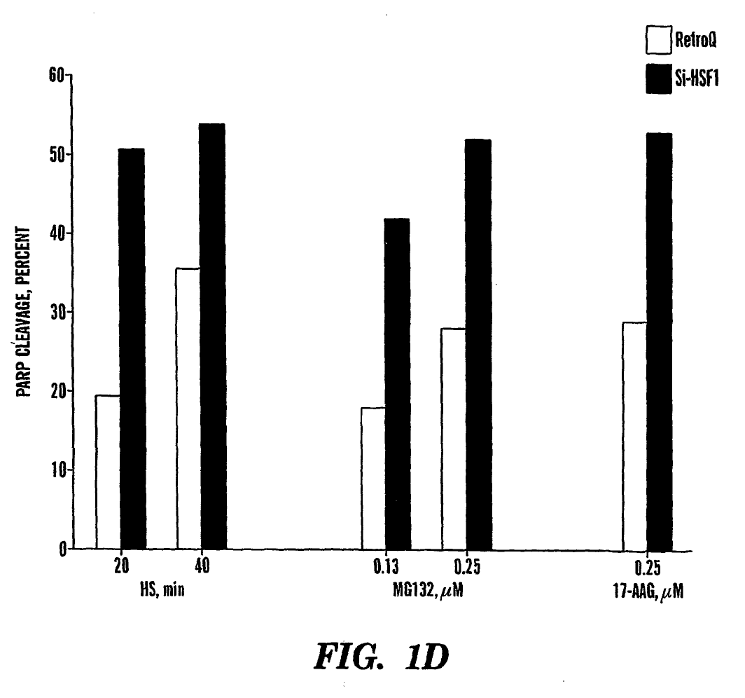

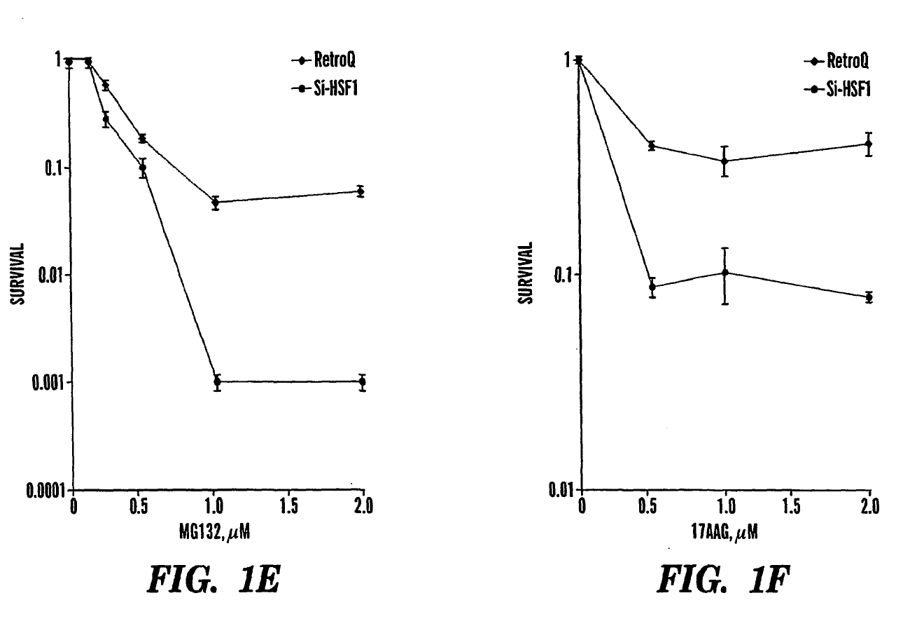

Novel classes of anti-cancer drugs, HSP90 and proteasome inhibitors, are potent inducers of the heat shock proteins. Since Hsps, especially Hsp72 and Hsp27 have strong anti-apoptotic activities, we hypothesized that inhibition of the heat shock response may promote the cytotoxic effects of these drugs, thus enhancing their anti-cancer activities. Thus, we tested whether prevention of induction of the Hsps can sensitize cancer cells to these drugs. Since expression of Hsps is regulated by the major heat shock transcription factor HSF1, depletion of HSF1 must make cells unable to induce Hsps, as was previously shown with the HSF1^{-/-} MEF cells 17. To deplete HSF1 in cancer cells, prostate carcinoma PC-3 cells were infected with retrovirus encoding siRNA against HSF1 (si-HSF1) or with a control virus (RetroQ). After a brief selection with puromycin, resistant populations were established, and at day 5 post-infection the levels of HSF1 in the si-HSF1 cells became undetectable, while the levels of HSF1 in RetroQ cells were not changed (FIG. 1A).

To confirm that depletion of HSF1 suppresses the stress response, PC-3 cells infected with either si-HSF1 or RetroQ were heated for 20, 30 and 40 min at 45° C. and then recovered for 24 hours at 37° C., and the levels of Hsp72 were measured by immunoblotting. In RetroQ cells, heat treatment led to a robust induction of Hsp72, while no changes of Hsp72 levels occurred in si-HSF1 cells, indicating the lack of the heat shock response.

When these cells were incubated with Hsp90 inhibitor 17-AAG (not shown) or proteasome inhibitor MG132 (FIG. 1B) for 24 hours, Hsp72 was strongly induced in RetroQ cells but not in si-HSF1 cells. Interestingly, the background levels of Hsp72 were not significantly altered upon depletion of HSF1, indicating that another transcription factor is responsible for maintaining elevated levels of Hsp72 in PC-3 cells. Such an alternative activator of the Hsps transcription in cancer cells could be an isoform of p63, as suggested previously¹⁸.

To test for the effects of suppression of the heat shock response on drug-sensitivity of cells, si-HSF1 cells were exposed to heat shock, MG132 or 17-AAG and degrees of apoptosis were tested by monitoring cleavage of PARP, a substrate of caspase-3. Exposure of control RetroQ cells to 45° C. for 20 min led to about 20% of PARP cleavage, while si-HSF1 cells showed dramatically increased PARP cleavage (about 50%). Similarly, effect was shown after 40 min of heat shock (FIG. 1D). These results with prostate carcinoma PC-3 cells are in line with previous results using non-cancerous primary MEF cells, demonstrating that HSF1-1-knockout cells are more sensitive to heat shock and an Hsp90 inhibitor than control MEFs (^{15,17}).

When PC-3 cells were exposed to the proteasomne inhibitor MG132 at concentration of 0.125 μM and 0.25 μM for 48 hours, PARP cleavage was about 2 times higher in si-HSF1 compared to control RetroQ cells (FIGS. 1C, and 1D). Similarly, depletion of HSF1 led to increase in PARP cleavage by 100% when PC-3 cells were exposed to the Hsp90 inhibitor 17-AAG at concentrations of 0.25 μM for 24 hours (FIG. 1D).

To assess the overall drug sensitivity of si-HSF1 and RetroQ PC-3 cells, we monitored the colony forming ability following exposure to drugs. After treatments with various concentrations of either MG132 or 17-AAG, cells were diluted in a medium and plated. After 10 days, colonies of surviving cells were stained and counted. When treated with MG132, at concentrations between 0.25 μM and 2 μM a very strong enhancement of sensitivity was seen with si-HSF1 cells (FIG. 1E). Similarly with 17-AAG, about 5-fold sensitization was seen at a wide range of concentrations (FIG. 1F).

Sensitization to apoptosis in response to anti-cancer drugs by suppression of the heat shock response was relatively specific, since we observed little or no sensitization by si-HSF1 in cells exposed to a distinct anti-cancer drug doxorubicin that does not activate the heat shock response.

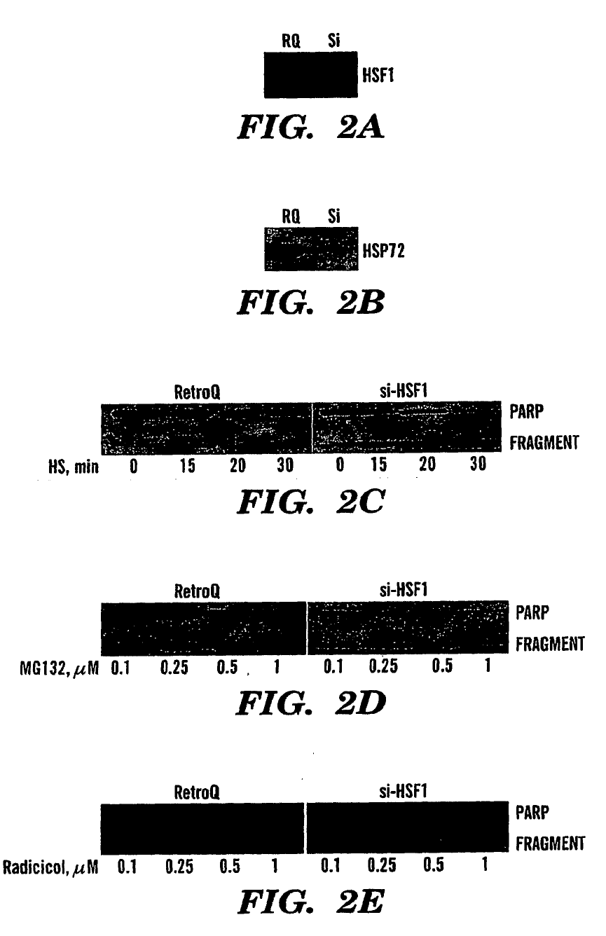

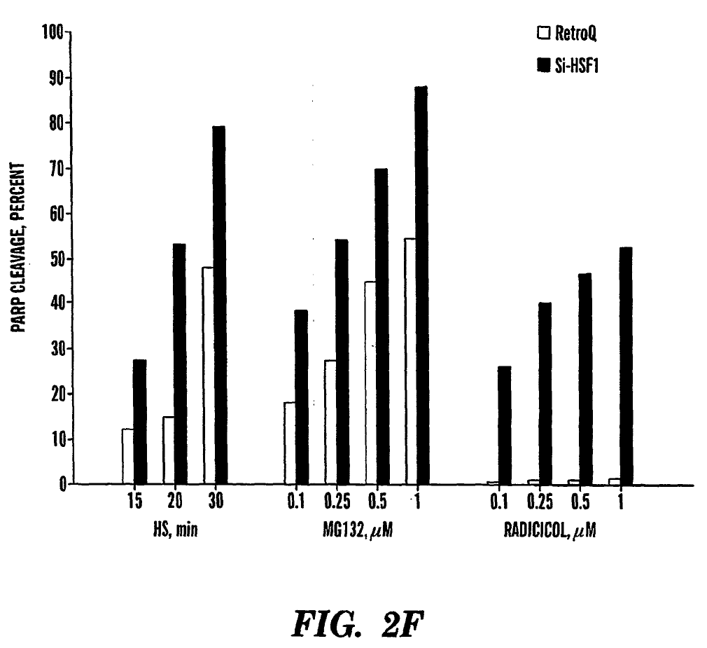

Sensitization to proteasome and Hsp90 inhibitors was seen when we tested additional cancer cells lines. In fact, depletion of HSF1 by treatment with siRNA in either a distinct prostate carcinoma line DU-145 or colon carcinoma HCT-116 cells led to suppression of the heat shock response and increased sensitivity to MG132 and 17-AAG (FIG. 2). Interestingly, in contrast to PC-3 and DU-145 cells, the high endogenous levels of Hsp72 in HCT-116 cells appeared to be dependent on HSF1. Depletion of HSF1 in these cells led to a dramatic reduction of the constitutive Hsp72 levels (FIG. 2B) and very strong sensitization to the drugs (FIGS. 2C, 2D, and 2E). Effects on sensitivity to Hsp90 inhibitor radicicol was most dramatic, since control cells were resistant to this drug, while si-HSF1 cells were quite sensitive, indicating that the resistance of original cells was due to the endogenous expression of one or several of the Hsps (FIG. 2E).

We screened for small molecules to identify inhibitors of induction of HSPs by these drugs.

Screening Chemical Libraries for Inhibitors of Hsps Induction.

We have developed a two-stage high throughput screen for inhibitors of Hsp expression. The first stage involves a cell-based screen for Hsp-mediated refolding of heat-denatured luciferase. Firefly luciferase expressed in mammalian cells is very sensitive to denaturation upon exposure of cells to severe but non-lethal heat insults. On the other hand, pretreatment of cells with milder heat shock leads to induction of Hsps which protect luciferase from further exposure to denaturing heat insults, and facilitates luciferase refolding. Therefore, exposure of cells to a potential inhibitor of Hsp induction would be predicted to suppress the protective effects of mild heat shock, and result in reduced luciferase activity after the second denaturing insult.

For the high throughput assay, we adopted CHO cells expressing luciferase under the control of TET-OFF™ promoter (CLONTECH). Cells were seeded in 96- or 384-well plates with medium that lacks tetracycline to allow induction of luciferase, and after 4 hours chemical compounds were added. Following overnight incubation at 37° C., cells were exposed to HS at 45° C. for 10 min to induce Hsps. After an additional six hours at 37° C., the plates were exposed to severe denaturing heat shock at 45° C. for 50 mim, followed by recovery for 70 min at 37° C. to allow luciferase refolding. The cells were then lysed and luciferase activity was measured. Exposure of cell to severe HS without pretreatment with mild heat shock led to unrepairable damage of luciferase. On the other hand, induction of Hsps after mild heat shock allowed rapid refolding of about 50% of luciferase after 70 min of recovery. Compounds that inhibit induction of Hsps must prevent luciferase refolding, and to select the inhibitors we established a cut-off line at 70% of inhibition of luciferase activity. As a counter-screen against toxic chemicals, the compounds were added to cells in multi-well plates, and kept for the duration of the entire experiment at 37° C. without exposure to heat shock. The cut-off line for toxicity was 30% inhibition of the luciferase activity in cells kept at 37° C.

Chemical compounds from the National Cancer Institute (NCI) Structural Diversity Set and Open Collection Set, as well as the Collection of Bioactive Compounds obtained from the Harvard Institute for Chemistry and Cell Biology (ICCB) were used for the screen. From about 20,000 chemicals, 40 compounds were found to inhibit luciferase refolding without showing significant toxicity in the luciferase test.

The compounds that were selected at the first step were directly tested for inhibition of Hsp induction by immunoblotting with an anti-Hsp72 antibody in the second step of the screening. Compounds at final concentration of 2 μM were added to CHO cells, and the cells were exposed to 45° C. for 10 min. and then incubated at 37° C. for 6 hours to allow accumulation of Hsp72. Cells were lysed and Hsp72 levels were measured.

Ten out of 40 originally selected suppressors of luciferase refolding were found to inhibit Hsp72 induction. Three of these compounds, including emetine, tubulosine, and NZ28, have significant structural similarity. Among these compounds, emetine and tubulosine, while passing the toxicity test at the first step of the screen, nevertheless showed toxicity in an apoptotic assay. In contract, NZ28 did not show toxicity in CHO cells, but rather slight growth inhibition.

In order to identify structural elements in this family of compounds that are critical for inhibition of induction of Hsps, we obtained fifteen compounds in the National Cancer Institute (NCI) chemical library using a similarity engine. These compounds were tested directly for inhibition of Hsp72 induction at concentrations between 2 and 10 μM (examples of these compounds are shown in Table 1).

Four compounds, including NZ71, NZ72, dihydroemetine, and isocephaeline, were found to inhibit Hsp72 induction by heat shock at this range of concentrations, while the rest of compounds were found to be inactive. Based on this analysis we defined the elements of the structure that are essential for the activity (highlighted). Furthermore, we were able to identify another compound that we called emunin (NZ71) that inhibited Hsp72 induction (Table 1), was non-toxic, and demonstrated very little cell growth inhibition.

Characterization of Novel Inhibitors of the Stress Response

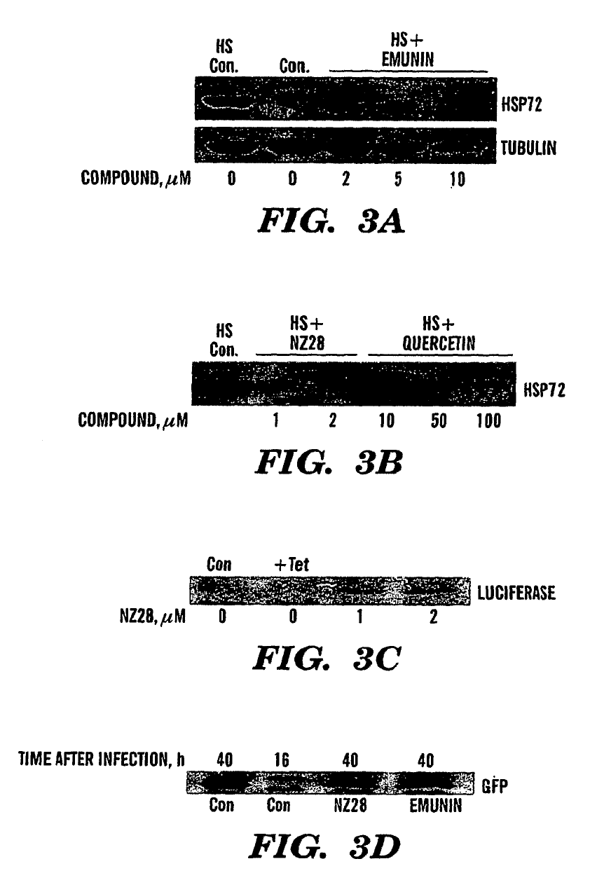

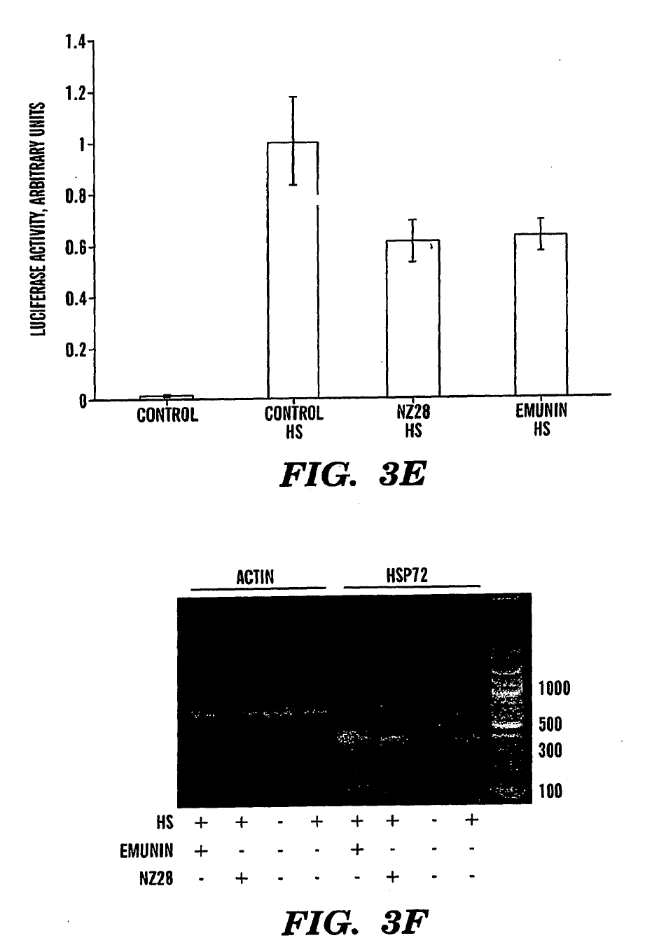

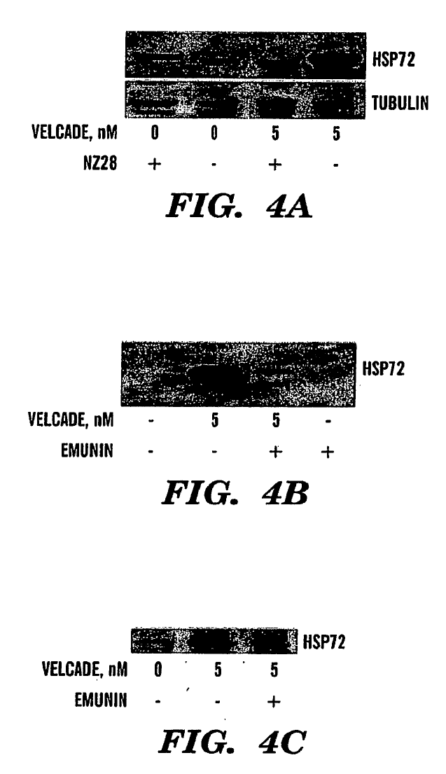

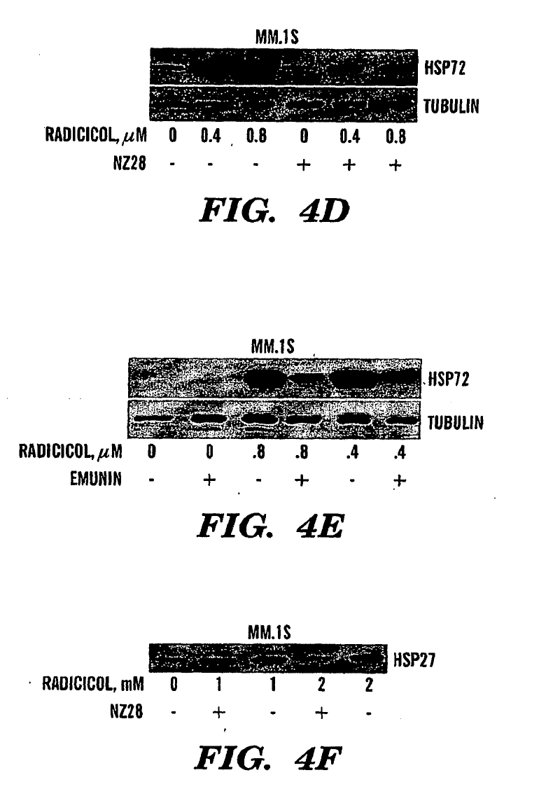

Both NZ28 and emunin were much more potent inhibitors of the stress response than a previously described inhibitor, the bioflavonoid quercetin, which works through inhibition of HSF1. In fact, in CHO cells the IC50 for inhibition of Hsps induction for quercetin was approximately 50 μM, while for emunin it was 5 μM and for NZ28 it was 1 μM FIGS. 3A and 3B). In all the following experiments emunin and NZ28 were used at concentrations of 10 and 2 μM, respectively. The inhibitory activities of the selected compounds were not limited to Hsp72. In fact we observed that induction of another heat shock protein Hsp27, which also has an anti-apoptotic activity, was strongly inhibited (see FIG. 4).

To assess the specificity of the selected inhibitors, we tested whether they have general inhibitory effects on protein synthesis. This was especially important since emetine, a structural analog of NZ28 and emunin, inhibits protein synthesis at concentrations above 10 μM. Therefore, we tested whether emunin and NZ28 can inhibit induction of two unrelated reporter proteins, luciferase and GFP, under the control of tet and CMV promoters, respectively. To investigate the effects on luciferase induction, the compounds were added to CHO cells, which express luciferase under the tet-off promoter, simultaneously with the removal of tetracycline, and after 24 hours cells were lysed, and the luciferase induction was assayed by immunoblotting with anti-luciferase antibody. Neither NZ28 (FIG. 3C) nor emunin (not shown) inhibited luciferase expression. To test for the effects of the compounds on the distinct reporter protein GFP, CHO cells were infected with retrovirus that encodes CMV-driven GFP gene. At 16 hours post-infection, i.e. at the time where no GFP is expressed yet, the compounds were added, and after additional 24 hours the GFP expression was assayed by immunoblotting with anti-GFP antibodies (FIG. 3D). No significant inhibition of GFP expression was seen in samples incubated with either NZ28 or emunin. These experiments indicate that these compounds do not cause general defects of transcription, translation, or protein degradation.

All known inhibitors of the heat shock response, including quercetin, stresgenin, and KNK437 inhibit activation of the transcription factor HSF1^{19 20 21} Therefore, we investigated whether the compounds that we selected affect activation of HSF1 and transcription of Hsps. To assess the effects of the compounds on the HSF1 activity, PC-3 cells were transiently transfected with a plasmid that encodes a reporter luciferase gene under the control of HSF1-activated Hsp70B promoter. On the second day after transfection emunin or NZ28 were added and after 5 hours, cells were exposed to heat shock at 45° C. for 10 min, followed by an overnight recovery at 37° C. in the presence of the inhibitors. Then cells were lysed, and luciferase activity was assayed (FIG. 3E). Although slight inhibition (about 40%) of luciferase induction by heat shock was seen with both NZ28 and emunin, the effect was markedly lower than almost complete inhibition of induction of Hsp72 under these conditions, showing that the major effect of the compounds is at the post transcription step.

To further assess whether the compounds affect transcription of Hsps, we performed RT-PCR using primers for Hsp72 (and β-actin as a control). Heat shock caused strong accumulation of Hsp72 mRNA, and neither NZ28 nor emunin reduced the mRNA levels (FIG. 3F). Therefore, in contrast to previously known inhibitors of the stress response, the newly selected compounds do not affect either synthesis or degradation of Hsp72 mRNA, but act at the post-transcriptional level.

In the next step, we investigated whether the compounds selected in the first screen can cause inhibition of the stress response in other cancerous and normal cell lines, including mouse fibroblasts (MEF), multiple myeloma MM.1S, prostate carcinoma PC-3, and colon carcinoma HCT-116 cells. Both NZ28 and emunin caused potent inhibition of Hsp72 induction after heat shock in all tested cell lines (Table 2), but the inhibitory effects were stronger with MM.1S than with other cells.

To test whether NZ28 and emunin can inhibit induction of Hsp72 in response to proteasome and Hsp90 inhibitors, MM.1S cells were exposed to proteasome inhibitors, VELCADE® or MG132 for 16 hours. Strong induction of HSP72 was seen under these conditions, while keeping the cells with either NZ28 or emunin during the course of the experiment almost completely inhibited HSP72 induction (FIGS. 4A and 4B). Similarly, these compounds inhibited induction of Hsp72 by the proteasome inhibitors in PC-3 cells (Table 2). These effects were seen 6 hours after addition of the proteasome inhibitors, but inhibition was relieved after 20 hours. Such transient inhibition of the stress response was nevertheless sufficient to sensitize these cells to proteasomne inhibitors.

Similarly, emunin and NZ28 inhibited induction of Hsp72 in response to Hsp90 inhibitors radicicol and 17-AAG. As with the proteasoine inhibitors, the response of multiple myeloma MM.1S cells (FIGS. 4D and 4E) to Hsp90 inhibitors was blocked by the selected compounds stronger than in PC-3 cells (Table 2). In fact, in PC-3 cells effects of NZ28 on inhibition of Hsps by 17-AAG were significant but transient, while emunin showed only weak inhibition of Hsp72 induction by 17-AAG (Table 2).

### NZ28 and Emunin Sensitize Cancer Cells to Proteasome and Hsp90 Inhibitors

As described above, inhibition of the heat shock response by depletion of HSF1 sensitized various cancer cells to proteasome and Hsp90 inhibitors. Here we used MM.1S cells to evaluate whether NZ28 and emunin that inhibit the stress response can sensitize cells to these novel classes of drugs. The degree of caspase-dependent apoptosis in response to proteasome and Hsp90 inhibitors was assessed by PARP cleavage.

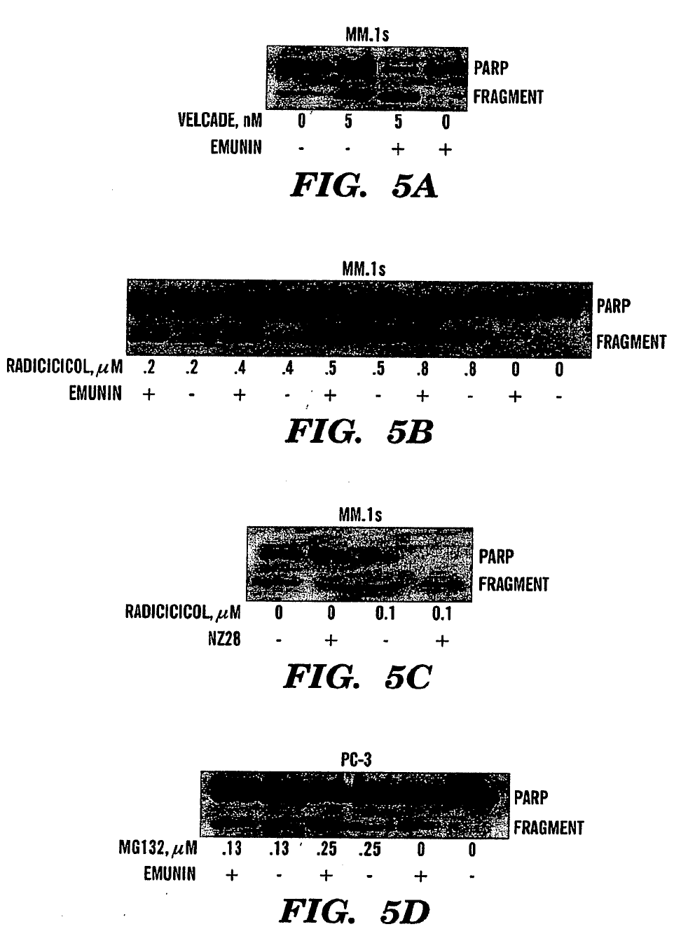

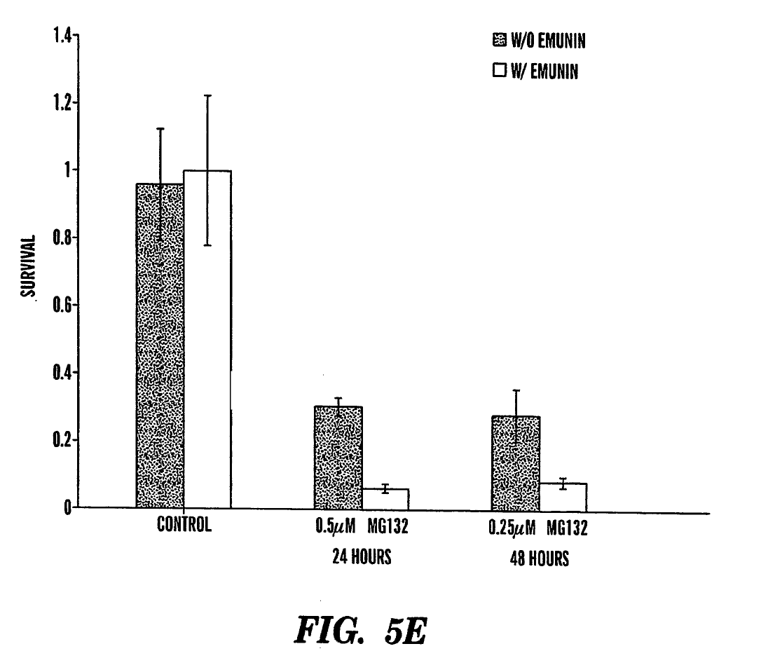

MM.1S cells were incubated with VELCADE® at a concentration of 5 nM with or without emunin. After overnight incubation, 30% cleavage was detected with VELCADE® alone, while incubation with emunin and VELCADE® together led to 70% of PARP cleavage (FIG. 5A). It is important to note that emunin alone did not cause PARP cleavage. To test for sensitization to Hsp90 inhibitors, MM.1S cells were incubated with radicicol with or without emunin for 24 hours. Sensitization of cells to radicicol was seen at a wide range of concentrations. For example, at 0.5 μM radicicol caused 10% of PARP cleavage in control cells, while incubation with both radicicol and emunin led to about 50% cleavage (FIG. 5B). NZ28 similarly sensitized MM.1S cells to radicicol (FIG. 5C).

Similar experiments were done with prostate carcinoma PC-3 cells. These cells were treated with a proteasome inhibitor, MG132 with or without emunin. After 2 days of incubation approximately 10% of PARP cleavage was detected with MG132 treatment alone, while incubation with both MG132 and emunin led to 50% of PARP cleavage (FIG. 5D). Similarly, addition of emunin very significantly reduced colony forming ability of cells treated with MG132 (FIG. 5E). At the same time, treatment with emunin alone did not affect the colony forming ability of these cells. On the other hand, we observed little sensitization of PC-3 cells by emunin to radicicol. That was probably because emunin was not efficient in inhibiting induction of Hsps in these cells upon exposure to Hsp90 inhibitors.

If emunin-mediated sensitization to proteasome inhibitors is related to suppression of induction of Hsps, we expected that this, compound would not further sensitize cells after depletion of HSF1. Accordingly, to test for the specificity of emunin, we investigated its effects on sensitivity of si-HSF1 PC-3 cells to MG-132. In contrast to RetroQ cells, no apparent sensitization was seen under these conditions, indicating that in fact the activity of emunin in cell sensitivity to the proteasome inhibitors is mostly related to suppression of the heat shock response.

Therefore, novel inhibitors of the stress response that we have identified may be used as sensitizers of cancer cells to novel classes of drugs, proteasome and Hsp90 inhibitors and could play a role in combination chemotherapy approaches.

FIG. 1 shows that two days after PC-3 cells were infected with retroviral vectors expressing siRNA to HSF1 (si-HSF1) or empty vector (RQ) cells were selected with puromycin (0.5 μg/ml). After two days of selection cells were exposed to stresses. FIG. 1A shows depletion of HSF1 by siRNA. HSF1 levels were tested by immunoblotting. FIG. 1B shows inhibition of Hsp72 induction by a proteasome inhibitor MG132 in cells after depletion of HSF1. si-HSF1 and RQ cells were exposed to MG132 at indicated concentrations, and after 16 hours incubation HSP72 levels were measured by immunoblotting. FIG. 1C shows depletion of HSF1 sensitizes cells to apoptosis caused by MG132. After 48 hours of incubation with MG132 apoptosis was measured by monitoring PARP cleavage. FIG. 1D shows quantification of apoptosis measured by PARP cleavage in cells exposed to heat shock, proteasome inhibitor MG132 and Hsp90 inhibitor 17-AAG. PARP cleavage 24 hr after heat shock or 17-AAG was quantified by Quantity One software (BIO-RAD). This experiment was repeated three times. Quantification of a typical experiment is presented. (FIGS. 1E and 1F show the effect of HSF1 depletion on overall clonogenic survival of cells exposed to MG132 (FIG. 1E) or 17-AAG (FIG. 1F) for 24 h.

FIG. 2 shows the effect of HSF1 depletion on sensitivity of HCT-116 cells to heat shock, proteasoine and HSP90 inhibitors. Infection of HCT-116 cells by retrovirus expressing si-HSF1 was done as described in FIG. 1. FIG. 2A shows expression of HSF1 in si-HSF1 cells. FIG. 2B shows expression of Hsp72 in si-HSF1 cells. FIGS. 2C, 2D, and 2E show effects of HSF1 depletion on sensitivity to apoptosis of cells exposed to heat shock at 45° C. for the indicated time (FIG. 2C), proteasome inhibitor MG132 (FIG. 2D), or HSP90 inhibitor radicicol, (FIG. 2E) at the indicate concentrations, and PARP cleavage was quantified after overnight incubation by Quantity One software (BIO-RAD) (FIG. 2F). This experiment was repeated three times. Quantification of a typical experiment is presented.

FIG. 3 shows the characterization of emunin and NZ28. Compounds were added to CHO cells at the indicate concentrations, and after 16 hour cells were exposed to heat shock at 45° C. for 10 min. After 6 hours cells were lysed and HSP72 levels were measured by immunoblotting. Control cells (“con.”) were not exposed to heat shock, and HS con. cells were exposed to heat shock but without compound. As a control for total protein tubulin antibody was used. FIG. 3A shows the effect of emunin on induction of Hsp72. FIG. 3B shows comparison of effects of NZ28 and quercetin on induction of Hsp72. FIGS. 3C and 3D show that the selected compounds do not affect general protein synthesis. Tetracycline was removed and NZ28 at concentration of 1 and 2 μM was added to CHO cells that express luciferase under the control of tet-regulated promoter. After 24 hours of incubation, luciferase was checked by immunoblotting with anti-luciferase antibody. As a positive control (“Con”), cells were kept without either tetracycline or NZ28, as a negative control cells were kept with tetracycline (FIG. 3C). CHO cells were infected with retrovirus encoding GFP under the control of CMV promoter. After 16 hours NZ28 (2 μM) or Emunin (10 μM) were added. As a control, no compounds were added. GFP levels were measured by immunoblotting with anti-GFP antibody. Con 16 h—beginning of GFP expression, the time of compounds addition. 40 h after infection—the time of full expression of GFP with or without compounds (FIG. 3D). Effects of NZ28 and Emunin on HSF1-dependent transcription. FIG. 3E shows PC-3 cells that were transfected with pGL.HSP70B plasmid, to express luciferase under the regulation of RSP70B gene. Two days after transfection cells were incubated with compounds and exposed to heat shock at 45° C. for 10 nm in. After overnight incubation luciferase assay was performed. HS control cells were exposed to heat shock without compounds. Control cells were not expose to HS. FIG. 3F shows PC-cells that were pre-incubated with Emunin 10 μM or NZ28 2 μM for five hours, and exposed to heat shock at 45° C. for 10 min. One hour after HS cells were lysed, RNA purified, and semi quantitative RT-PCR was performed as described in Materials and Methods. βActin mRNA expression was tested as a control.

FIG. 4 shows that emunin and NZ28 inhibit HSP72 and HSP27 induction by proteasome and HSP90 inhibitors.

MM.1S cells were incubated with proteasome inhibitor VELCADE® or with HSP90 inhibitor Radicicol at the indicated concentrations with or without compounds. 10 μM Emunin or 2 μM NZ28 were added 5 hours before the treatments with the inhibitors. HSP72 and HSP27 levels were measured after overnight incubation. Immunoblotting with anti-tubulin antibody was used as a loading control.

FIG. 5 shows that emunin and NZ28 sensitize MM.1S and PC-3 cells to proteasomne and HSP90 inhibitors.

In all the cases, compounds were pre-incubated 5 hours before the treatments. Apoptosis was measured by PARP-cleavage. FIG. 5A shows MM.1S cells that were incubated with 5 nM of proteasome inhibitor VELCADE® with or without 10 μM emunin. FIG. 5B shows MM.1S cells that were incubated with HSP90 inhibitor radicicol with or without 10 μM emunin for 24 hours. FIG. 5C shows MM.1S cells that were incubated with 0.1 μM of HSP90 inhibitor radicicol for 48 h with or without 2 μM NZ28. FIG. 5D shows PC-3 cells that were incubated with, 0.13 μM or 0.25 μM of proteasome inhibitor MG132 for 48 h. FIG. 5E shows the effect of emunin on clonogenic survival of PC-3 cell incubated with 0.5 and 0.25 μM of proteasome inhibitor MG132 for 24 hours or 48 hours, respectively.

Materials and Methods

Cell cultures and treatments: MM.1S myeloma, PC-3, and DU-145 prostate carcinoma cells were grown in RPMI-1640 medium with 10% fetal bovine serum FBS, HCT-116 colon carcinoma cells were grown in McCoy medium with 10% FBS; MEF cells and CHO-Luciferase TET-OFF cells were grown in Dulbecco modified Eagle medium (DMEM) with 10% (FBS); for CHO cells gentamycin (100 μg/ml), hygromycin (100 μg/ml) and tetracycline (1 μg/ml) were added. All cells were grown at 37° C. in an atmosphere of 5% CO2.

Chemical compounds libraries were provided by the NCI and ICCB; emetine and quercetin were from Sigma; all compounds were diluted in dimethyl sulfoxide (DMSO) as 10 mM stock solutions. MG132, radicicol, doxorubicin, 17-AAG were from BIOMOL International L.P., Plymouth Meeting, Pa.; cis-platinum was from Sigma-Aldrich Co., St Louis, Mo.). Cells were exposed to heat shock by immersing plates or dishes wrapped with parafilm in a water bath at the desired temperatures (+/−0.1° C.).

Small interfering RNA (si-RNA), retrovirus infection and transfection: For knocking down HSF1 in PC-3, DU-145, HCT-116 cells we used RNAi-READY-pSIREN-RetroQ vector with puromycin resistance (CLONTECH Laboratories Inc., a Takara Bio Company). The sequences of human HSF1 gene that was selected as a target for RNA interference was 5′-TATGGACTCCAACCTGGATAA-3′ (SEQ ID NO 1).

For production of retroviruses, 293T cells were co-transfected with plasmids expressing retroviral proteins Gag-Pol, G (VSVG pseudotype), or GFP, or our construct using LIPOFECTAMINE™ 2000 (INVITROGEN™); supernatants containing the retrovirus were collected 48 h after transfection and kept at −70° C. For infection, cells were incubated with two times diluted retrovirus supernatant and 10 μg/ml polybrene (Sigma-Aldrich, Co., St Louis, Mo.) overnight, washed and selection with puromycin was started 48 h after infection.

PGL.hsp70B luciferase promoter regulated by HSP70B gene was described previously 24. PC-3 cells were transfected with pGL.hsp70B plasmid (1 μg) with 6 μl of GENEPORTER™ (GTS Inc., San Diego, Calif.) in 35 mm dishes, and 48 hr later they were used for experiments.

High-throughput screening: Chemical compounds from various libraries were dissolved in DMSO at concentration of 1 mM and distributed in 384-well master plates. CHO cells were plated in 384-well white bottom plate at 2500 cells per well in 50 μL media without tetracycline using liquid handling robots (BIO-TEK® PRECISION™ 2000 robot). After cells attached, 100 nl of chemical compounds were transferred from master plates to assay plates using an automated pin-based compound transfer robot to final concentration of 2 μM. In each plate one column was without chemical compound but with DMSO as a negative control. Four plates were prepared for each set of compounds, two plates for the inhibitor assays and two for toxicity assays. Sixteen hr after incubating cells with compounds plates were immersed in 45° C. water bath for 10 min, kept for 6 hr at 37° C., than exposed again to 45° C. for 50 min, and after 70 min at 37° C. luciferase assay was performed. For luciferase assay cells were washed twice with PBS and lysed with cell lysis reagent (Promega Corporation, Madison, Wis.) 10 μL for each well in 384 well plate. Samples were frozen at −70° C. and thawed before checking luciferase activity. 20 μL for 384 well plate of luciferase reagent (Promega Corporation, Madison, Wis.) were dispensed per well and luminescence was read by luminometer (BIO-RAD Laboratories, Hercules, Calif., or ANALYST® LjL BIOSYSTEMS, Sunnyvale, Calif.).

Clonogenic Assay: To measure clonogenic survival, after treatments cells were counted and plated on 100 mm dishes at appropriate numbers. Ten days later colonies were stained with 0.5% crystal violet in 70% ethanol. Quantification of colonies was made by AXIOVISION™ 4.3 program (Carl Zeiss AG, Germany).

Western blotting: Cells were washed with PBS and lysed in 80 μl of lysis buffer (40 mM HEPES, pH 7.5; 50 mM KCL; 1% TRITON-X-100; 2 mM DTT; 1 mM Na3VO4; 50 mM β-glycerolphosphate; 50 mM NaF; 5 mM EDTA; 5 mM EGTA; 1 mM PMSF; 5 μg/ml of each: leupeptine, pepstatine A, aprotinin) per 35 mm dish. Total protein concentration was measured by BIO-RAD protein assay reagent and the samples were diluted with lysis buffer to achieve equal protein concentration at all the samples. To measure of PARP cleavage cells were washed with PBS and lysed in 120 μl lysis buffer per 35 mm dish (4M urea, 10% glycerol, 2% SDS, 5% 2-mercaptoethanol and 0.01% bromophenol blue).

Following antibodies were used for immunoblotting: SPA-901 for HSF1; SPA-810 for HSP72; SPA-800 for Hsp27 (all from NVENTA, Nventa Biopharmaceuticals Corporation), anti-PARP (BD Biosciences, San Jose, Calif.), anti-luciferase (Sigma-Aldrich, Co., St Louis, Mo.), anti-GFP (ClonTech); anti-β-Actin (Sigma-Aldrich, Co., St Louis, Mo.), anti-γ-tubulin (Santa-Cruz). After incubation with a primary antibody, secondary antibodies conjugated with peroxidase were visualized with ECL system (Amersham).

Semi-Quantity RT-PCR: Total RNA was isolated from cells at 70% confluency from 35 mm dish using TRIZOL® reagent (INVITROGEN™, Corporation). After spectrophotometric quantification of RNA (BIOPHOTOMETER, EPPENDORF® AG, Germany) reverse transcription was perform using RETROSCRIPT® kit (AMBION® Inc., Austin, Tex.) following factory protocol. Briefly, 1 μg RNA was mixed with 2 μl oligodT primer for denaturation and kept on ice. dNTPmix, RNase inhibitor and 100 U of reverse transcriptase were added to the RNA denaturated mixture and the reaction was carried out for one hour at 43° C. PCR was performed in 25 μl reaction mixture containing 2 μl RT reaction, 0.4 μM dNTP mix, 1 U Tag-DNA-polymerase (New England BIOLABS®, Inc. Ipswich, Mass.) and 1.5 μM of each primer pair (HSP70A or β-Actin). For HSP70A the forward primer was: 5′-TGTTCCGTTTCCAGCCCCCAA-3′ (SEQ ID NO 2) and the reverse was: 5′-GGGCTTGTCTCCGTCGTTGAT-3′ (SEQ ID NO 3) to give 359 bp. β-Actin forward primer was 5′-CAGCTCACCATGGATGATGAT-3′ (SEQ ID NO 4) and the reverse was: 5′-CTCGGCCGTGGTGGTGAAGCT-3′ (SEQ ID NO 5) to give 626 bp. Amplification by PCR instrument (MASTERCYCLER® gradient, EPPENDORF® AG, Germany) was performed by 3 minutes at 95° C. for denaturation, and 30 cycles of 94° C. for 30 sec, 58° C. for 30 sec and 72° C. for 60 sec. The final extension was carried at 72° C. for 5 min. RT-PCR products were analyzed by running samples on 1.5% agarose gel in the presence of ethidium bromide, and visualized the product under UV light.

Table 1 shows the effect of selected compounds on Hsp72 induction and toxicity in CHO cells.

TABLE 1
EFFECT OF SELECTED COMPOUNDS ON HSP72 INDUCTION
AND TOXICITY IN CHO CELLS

| | | IC50 HSP72 | |
|---|---|---|---|
| | | Induction | IC50 |
| | | Inhibition | toxicity, |
| Name | Structure | (CHO cells), μM | μM |
| Emetine,RN: 5884-45-7 | 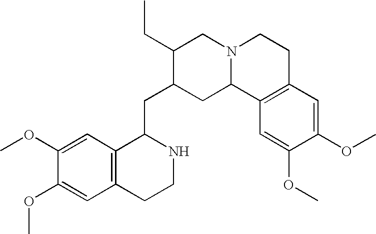 | <0.25 | <0.25 |
| Isocephaeline(Emetan-6′-ol, 7′,10,11-trimethoxy-,(1′beta)-)NCS-32944 | 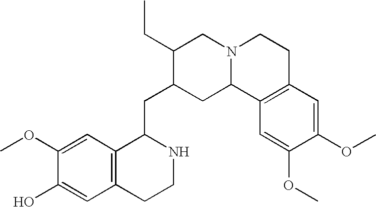 | <0.5 | <0.5 |
| dehydroemetineNCS-129414 | 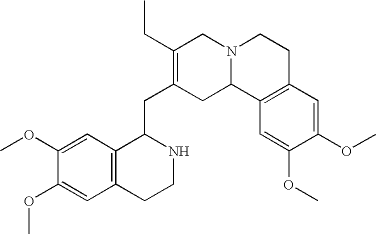 | <0.5 | <0.5 |
| NZ60NCS-134757 | 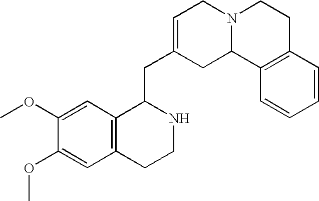 | >10 | <2 |
| NZ28NCS-134754 | 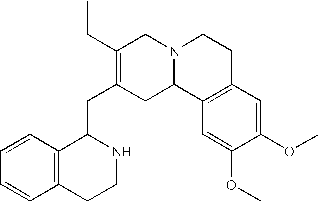 | 1 | 5 |
| NZ62NCS-134759 | 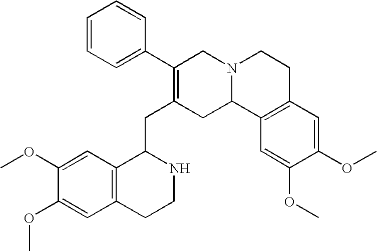 | >10 | <2 |
| NZ71emuninNCS-113238 | 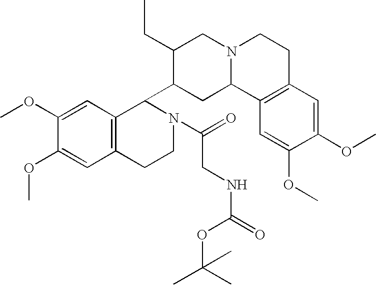 | 5 | >10 |
| NZ61NCS-134758 | 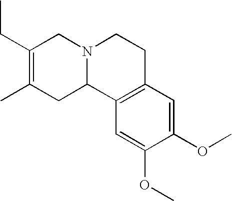 | >10 | <2 |
| NZ54NCS-118072 | 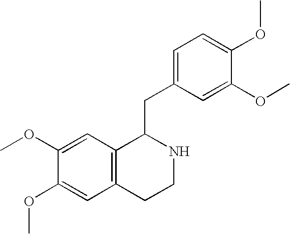 | >10 | <2 |
| NZ50NCS-10105 | 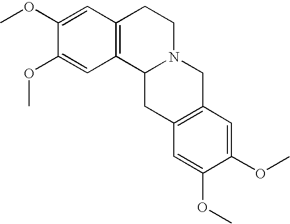 | >10 | <2 |
| tubulosineNCS-131547 | 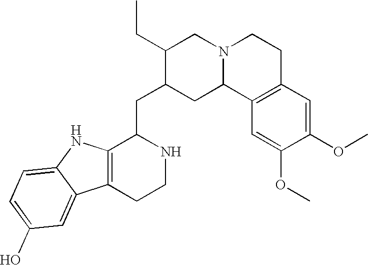 | 0.5 | 0.25 |
| NZ72NCS-131548 | 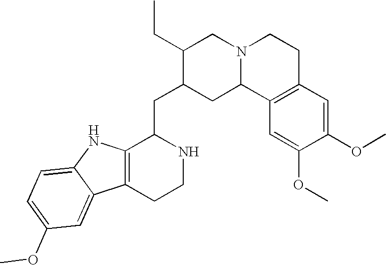 | 2 | >2 |

Table 2 shows HSP72 inhibition by NZ28 and emunin.

TABLE 2
HSP72 INHIBITION BY NZ28 AND EMUNIN

| | | | HSP72 | HSP72 |
|---|---|---|---|---|
| | | | Inhibition, | inhibition, |
| | | Stress | by NZ28 | by Emunin |
| Cell line | Stress | conditions | (2 μM), % | (10 μM), % |
| CHO | Heat Shock | 45° C., 10 min | 99 | 95 |
| MM.1s | | 45° C., 4 min | 100 | 90 |
| PC-3 | | 45° C., 20 min | 85 | 80 |
| MM.1s | Proteasome | VELCADE ®, | 100 | 95 |
| | Inhibitor | 5 nM | | |
| PC-3 | | MG132, 1 μM | 90 | 80 |
| MEF | | VELCADE ®, | 100 | — |
| | | 10 nM | | |
| MM.1s | HSP90 | Radicicol, | 90 | 90 |
| | Inhibitor | 0.8 μM | | |
| PC-3 | | 17-AAG, 2 μM | 70 | 60 |
| MEF | | Radicicol, | 100 | — |
| | | 0.2 μM | | |

### REFERENCES

1. Adams, J. The development of proteasome inhibitors as anticancer drugs. Cancer Cell 5, 417-21 (2004).
2. Mitsiades, N. et al. Molecular sequelae of proteasome inhibition in human multiple myeloma cells. PNAS 99, 14374-14379 (2002).
3. Chauhan, D., Hideshima, T., Mitsiades, C., Richardson, P. & Anderson, K. C. Proteasome inhibitor therapy in multiple myelomna. Mol Cancer Ther 4, 686-692 (2005).
4. Neckers, L. Hsp90 inhibitors as novel cancer chemotherapeutic agents. Trends Mol Med 8, S55-61. (2002).
5. Hostein, I., Robertson, D., DiStefano, F., Workman, P. & Andrew Clarke, P. Inhibition of Signal Transduction by the Hsp90 Inhibitor 17-Allylamino-17-demethoxygeldanamycin Results in Cytostasis and Apoptosis. Cancer Res 61, 4003-4009 (2001).
6. Fujita, N., Sato, S., Ishida, A. & Tsuruo, T. Involvement of Hsp90 in signaling and stability of 3-phosphoinositide-dependent kinase-1. J Biol Chem 277, 10346-53 (2002).
7. Broemer, M., Krappmann, D. & Scheidereit, C. Requirement of Hsp90 activity for IkappaB kinase (IKK) biosynthesis and for constitutive and inducible IKK and NF-kappaB activation. Oncogene 23, 5378-86 (2004).
8. Gabai, V. L. & Shennan, M. Y. Hsp72 and Cell Signalling, in Molecular Chaperones and Cell Signalling (ed. Henderson B., P.A.) 144-159 (Cambridge University Press, Cambridge, 2005).
9. Mosser, D., Morimoto, R I. Molecular chaperones and the stress of oncogenesis. Oncogene 23, 2907-2918 (2004).
10. Ciocca, D., Calderwood, S K. Heat shock proteins in cancer: diagnostic, prognostic, predictive, and treatment implications. Cell Stress & Chaperones 10, 86-103 (2005).
11. Beere, H. M. ‘The stress of dying’: the role of heat shock proteins in the regulation of apoptosis. J Cell Sci 117, 2641-2651 (2004).
12. Chauhan, D. et al. Blockade of Hsp27 Overcomes Boltezomib/Proteasome Inhibitor PS-341 Resistance in Lymphoma Cells. Cancer Res 63, 6174-6177 (2003).
13. Gabai, V. L., Budagova, K. R. & Shenran, M. Y. Increased expression of the major heat shock protein Hsp72 in human prostate carcinoma cells is dispensable for their viability but confers resistance to a variety of anticancer agents. 24, 3328-3338 (2005).
14. Meriin, A. B. et al. Protein damaging stresses activate JNK via inhibition of its phosphatase: A novel pathway controlled by Hsp72. Mol. Cell. Biol. 19, 2547-2555 (1999).
15. Bagatell, R. et al. Induction of a Heat Shock Factor 1-dependent Stress Response Alters the Cytotoxic Activity of Hsp90-binding Agents. Clin Cancer Res 6, 3312-3318 (2000).
16. Asea, A., Ara, G, Teicher, B A, Stevenison, M A, Calderwood, S K. Effects of the flavonoid drug quercetin on the response of human prostate tumours to hypertlhermia in vitro and in vivo. Int J Hypelliermia 17, 347-356 (2001).
17. McMillan, D. R., Xiao, X., Shao, L., Graves, K. & Benjamin, I. J. Targeted disruption of heat shock transcription factor 1 abolishes thermotolerance and protection against heat-inducible apoptosis. J Biol Chem 273, 7523-7528 (1998).
18. Wu, G. et al. ΔNp63α Up-Regulates the Hsp70 Gene in Human Cancer. Cancer Res 65, 758-766 (2005).
19. Hosokawa, N. et al. Inhibition of the activation of heat shock factor in vivo and in vitro by flavonioids. Mol Cell Biol 12, 3490-S (1992).
20. Akagawa, H., Takano, Y, Ishii, A, Mizuno, S, Izui, R, Sameshima, T, Kawamura, N, Dobashi, K, Yoshioka, T. Stresgenin B, an inhibitor of heat-induced heat shock protein gene expression, produced by Streptomyces sp. AS-9. J Antibiot 52, 960-970 (1999).
21. Ollislai, K., Takahashi, A, Yokota, S, Olinishi, T. Effects of a heat shock protein inhibitor KNK437 on heat sensitivity and heat tolerance in human squamous cell carcinoma cell lines differing in p53 status. Int J Radiat Biol. 80, 607-614 (2004).
22. Khialeque, M., Bharti, A, Sawyer, D, Gong, J, Benjamin, I J, Stevenson, M A, Calderwood, S K. Induction of heat shock proteins by heregulin beta1 leads to protection from apoptosis and anchorage-independent growth. Oncogene, in press (2005).
23. Pettit, G. R. G., Shyami Kirti. Antineoplastic agents. XX. Structural biochemistry. VII. Synthesis of 2′(N′-bis(2-chloroethyl)glycyltryptophyl)emetine and related peptides. Canadian Journal of Chemistry 45, 1561-1566 (1967).
24. Chem, C., Xie, Y., Stevenson, M. A., Auron, P. E. & Calderwood, S. K. Heat Shock Factor 1 Represses Ras-induced Transcriptional Activation of the c-fos Gene. J. Bol. Chem. 272, 26803-26806 (1997).

All references described herein and throughout the specification are incorporated by reference in their entirety.

We claim:

1-28. (canceled)

29. A method of treating cancer comprising administering to a subject with cancer a combination of a heat shock response inactivating agent and an anti-cancer agent.

30. The method of claim 29, wherein the heat shock response inactivating agent is a heat shock transcription factor Hsf1 inhibitor.

31. The method of claim 29, wherein the anti-cancer agent is an agent that causes DNA damage.

32. The method of claim 31, wherein the agent that causes DNA damage is selected from the group consisting of radiation, doxorubicin and camptothecin.

33. The method of claim 29, wherein the anti-cancer agent induces a heat shock response in cells.

34. The method of claim 33, wherein the anti-cancer agent is a heat shock protein 90 (Hsp90) inhibitor.

35. The method of claim 34, wherein the Hsp90 inhibitor is selected from the group consisting of 17-allylamino-17-demethoxygeldanamycin (17-AAG), 17-NN-Dimethyl Ethylene Diamine-Geldanamycin (17-DMAG) and radicicol.

36. The method of claim 29, wherein the anti-cancer agent is a proteasome inhibitor.

37. The method of claim 36, wherein the proteasome inhibitor is bortezomib or N-carbobenzoxyl-Leu-Leu-Leucinal.

38. The method of claim 29 or 30, wherein the heat shock response inactivating agent contains a 2H-benzo[a]quinolizine tricyclic ring.

39. The method of claim 29 or 30, wherein the heat shock response inactivating agent is NZ28 (NCS-134754) with a formula: 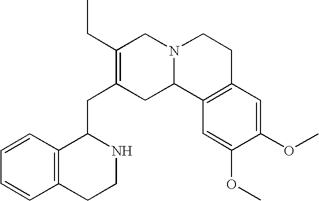
or emunin (NCS-113238) with a formula:

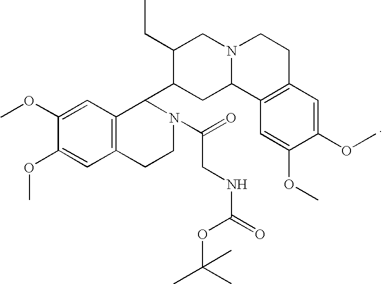

40. A method for treating cancer comprising administering to a subject with cancer cells a combination treatment comprising an effective heat shock response inactivating amount of a heat shock response inactivating agent and an anti-cancer agent.

41. The method of claim 40, wherein the heat shock response inactivating agent is a heat shock transcription factor Hsf1 inhibitor.

42. The method of claim 40, wherein the anti-cancer agent is an agent that causes DNA damage.

43. The method of claim 42, wherein the agent that causes DNA damage is selected from the group consisting of radiation, doxorubicin and camptothecin.

44. The method of claim 40, wherein the anti-cancer agent induces a heat shock response in cells.

45. The method of claim 44, wherein the anti-cancer agent is a heat shock protein 90 (Hsp90) inhibitor.

46. The method of claim 45, wherein the Hsp90 inhibitor is selected from the group consisting of 17-allylamino-17-demethoxygeldanamycin (17-AAG), 17-NN-Dimethyl Ethylene Diamine-Geldanamycin (17-DMAG) and radicicol.

47. The method of claim 40, wherein the anti-cancer agent is a proteasome inhibitor.

48. The method of claim 47, wherein the proteasome inhibitor is bortezomib or N-carbobenzoxyl-Leu-Leu-Leucinal.

49. The method of claim 40 or 41, wherein said heat shock response inactivating agent contains a 2H-benzo[a]quinolizine tricyclic ring.

50. The method of claim 40 or 41, wherein said heat shock response inactivating agent is NZ28 (NCS-134754) with a formula 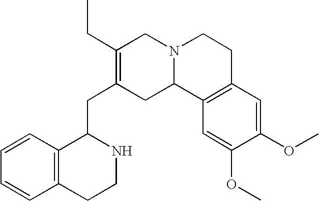
or emunin (NCS-113238) with a formula

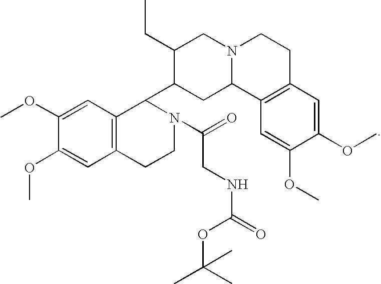

51. The method of claim 40, wherein the anti-cancer agent is a heat shock protein 90 (HSP90) inhibitor or a proteasome inhibitor.

52. The method of claim 40, wherein the amount of anti-cancer agent in the combination is reduced by 5-10% when compared to a treatment without a heat shock response inactivating agent.

53. The method of claim 40, wherein the amount of anti-cancer agent in the combination treatment is reduced by 10-20% when compared to a treatment without a heat shock response inactivating agent.

54. The method of claim 40, wherein the amount of anti-cancer agent in the combination treatment is reduced by 20% when compared to a treatment without a heat shock response inactivating agent.

55. The method of claim 40, wherein the amount of anti-cancer agent in the combination treatment is reduced by 25-50% when compared to a treatment without a heat shock response inactivating agent.
